(19) United States
(12) Patent Application Publication
(10) Pub. No.: US 2023/0039487 A1
(43) Pub. Date: Feb. 9, 2023

(54) BCMA-binding antibody and use thereof

(71) Applicant: SHENZHEN FEIPENG BIOLOGICAL THERAPY CO. LTD, Shenzhen, Guangdong (CN)

(72) Inventors: Lijun YE, Shenzhen, Guangdong (CN); Ting FENG, Shenzhen, Guangdong (CN); Jiamei ZHANG, Shenzhen, Guangdong (CN); Xianjin WANG, Shenzhen, Guangdong (CN); Baolei WANG, Shenzhen, Guangdong (CN); Liang PENG, Shenzhen, Guangdong (CN); Lisheng LU, Shenzhen, Guangdong (CN)

(21) Appl. No.: 17/785,419

(22) PCT Filed: Dec. 16, 2020
(86) PCT No.: PCT/CN2020/136725

§ 371 Date: Jun. 15, 2022

(30) Foreign Application Priority Data
Dec. 17, 2019 (CN) 201911298620.7

(51) Int. Cl. C07K 16/28; C12N 15/86; A61P 35/00; A61K 35/17

(52) U.S. Cl. CPC C07K 16/2878; C12N 15/86; A61P 35/00; A61K 35/17; C07K 2317/565; C07K 2317/567; C07K 2319/33; C07K 2319/03; C07K 2319/02; C12N 2740/15043; A61K 2039/5156

(57) ABSTRACT

Provided is an antibody, which is capable of specifically binding to a B-cell maturation antigen (BCMA). The provided BCMA antibody is capable of specifically binding to an extracellular fragment of the BCMA and has excellent affinity and specificity; and the antibody is a functional antibody and has the activity blocking binding of the BCMA with its ligand APRIL. Immune cells constructed based on the antibody has an excellent specific killing function for a BCMA-positive tumor cell.

### TECHNICAL FIELD

The present disclosure relates to the field of biomedicine, in particular to an antibody capable of specifically binding to a B-cell maturation antigen (BCMA), a chimeric antigen receptor containing the antibody or an immune cell containing the antibody.

### BACKGROUND

Multiple myeloma (MM) is a common malignant hematological disease, accounting for 2% of all cancer death. According to Global data 2019 statistics, the number of MM cases worldwide is 353,890 in 2017, and it is expected to reach 555,243 in 2027. Its main symptom is the indefinite proliferation of a plasma cell in bone marrow, thereby leading to osteonecrosis. At present, treatments for MM are mainly symptomatic treatment, chemotherapy, radiotherapy or stem cell transplantation, but the nearly 100% recurrence rate makes the treatment of the disease extremely difficult.

BCMA(B-cell maturation antigen) is a member of a TNF superfamily receptor (TNFRSF17, a type-III transmembrane protein, the full-length is 185 amino acids, and the extracellular fragment is 54 amino acids). It is specifically highly expressed on the surface of the plasma cell and multiple myeloma cell; it is not expressed in a memory B cell, a hematopoietic stem cell and other normal tissue cells. Its function is to regulate the activation, differentiation and transformation of the B cell into the plasma cell and prolongs the lifespan of the plasma cell together with the same family receptors TACT, BAFFR and ligands APRIL/BAFF; during the differentiation process of the B cell into the plasma cell, the expression of BCMA on the cell surface is up-regulated, a mouse lacking BCMA has the normal numbers of healthy and normal-looking B cells, but the life cycle of the plasma cell is shortened.

At present, the treatment for a patient with the multiple myeloma is poor in effect, high in cost, and long in cycle. Because BCMA is specifically highly expressed on the surface of the plasma cell and myeloma cell, BCMA is an ideal target for the treatment of the multiple myeloma. Existing clinical results show that immune cell therapy for the patient with the multiple myeloma is significantly better than the chemotherapy and radiotherapy; and in view of this, a functional antibody targeting BCMA and a derived immune cell therapy product thereof are urgently needed in the field.

### SUMMARY

The present disclosure relates to an antibody, and it is capable of specifically binding to BCMA, and selected from a) and/or b):

a) including heavy chain complementarity determining regions CDR-VH1, CDR-VH2, and CDR-VH3 of which amino acid sequences are as shown in SEQ ID NO: 1˜3, and light chain complementarity determining regions CDR-VL1, CDR-VL2, and CDR-VL3 of which amino acid sequences as shown in SEQ ID NO: 4˜6; and

b) including heavy chain complementarity determining regions CDR-VH1, CDR-VH2, and CDR-VH3 of which amino acid sequences are as shown in SEQ ID NO: 19˜21, and light chain complementarity determining regions CDR-VL1, CDR-VL2, and CDR-VL3 of which amino acid sequences as shown in SEQ ID NO: 22˜24.

The present disclosure further relates to a chimeric antigen receptor containing the antibody.

The present disclosure further relates to a nucleic acid for expressing the antibody and the chimeric antigen receptor, a vector and a host cell.

The present disclosure further relates to an immune cell, and it contains the chimeric antigen receptor as described above.

The present disclosure further relates to a pharmaceutical composition, and it includes the antibody as described above or the immune cell as described above, and one or more of a pharmaceutically acceptable excipient, diluent or carrier.

Compared with an existing technology, the beneficial effects of the present disclosure are as follows.

(1) The BCMA antibody provided by the present disclosure may specifically bind to the extracellular fragment of BCMA, and has the excellent affinity and specificity (it basically does not bind to other antigens on the surface of a cell membrane); and the antibody is afunctional antibody, and it has the activity of blocking the binding of BCMA to its ligand APRIL.

(2) The immune cell constructed on the basis of the antibody and capable of expressing the chimeric antigen receptor has a very good specific killing function for a BCMA-positive tumor cell.

### BRIEF DESCRIPTION OF THE DRAWINGS

In order to more clearly describe specific embodiments of the present disclosure or technical schemes in an existing technology, drawings required in the description of the specific embodiments or the existing technology are briefly introduced below. Apparently, the drawings in the following description are some embodiments of the present disclosure. For those of ordinary skill in the art, other drawings may also be obtained according to these drawings without creative work.

FIG. 1 shows affinity of a BCMA antibody (5E2, 5F4) for BCMA detected by an enzyme-linked immunosorbent assay (ELISA) in an embodiment of the present disclosure.

FIG. 2 shows affinity of the BCMA antibody (5E2, 5F4) for BCMA detected by Fortebio in an embodiment of the present disclosure.

FIG. 3 shows affinity of the BCMA antibody (5E2, 5F4) for BCMA detected by fluorescence-activated cell sorter (FACs) in an embodiment of the present disclosure.

FIG. 4 shows a result of a competitive binding experiment of the BCMA antibody (5E2, 5F4) and a BCMA ligand APRIL in an embodiment of the present disclosure.

FIG. 5 shows a specificity detection result of the BCMA antibody (5E2, 5F4) in an embodiment of the present disclosure.

FIG. 6 is a structure schematic diagram of a C11D5.3CAR plasmid used in an embodiment of the present disclosure.

FIG. 7 is a structure schematic diagram of a 5E2CAR plasmid used in an embodiment of the present disclosure.

FIG. 8 shows an experimental result of a CAR positive rate of flow cytometry detection of a chimeric antigen receptor T-cell (CART) in an embodiment of the present disclosure.

FIG. 9 shows a detection result of target cell apoptosis after CART and a target cell are co-cultured for 8 h in an embodiment of the present disclosure.

FIG. 10 shows a detection result of IL-2 after the CART cell and the target cell are co-cultured for 8 h in an embodiment of the present disclosure.

FIG. 11 shows a detection result of IFNγ after the CART cell and the target cell are co-cultured for 8 h in an embodiment of the present disclosure.

FIG. 12 shows an affinity result of a BCMA humanized antibody for a BCMA antigen detected by ELISA in an embodiment of the present disclosure.

FIG. 13 shows an affinity result of the BCMA humanized antibody for the BCMA antigen detected by Fortebio in an embodiment of the present disclosure.

FIG. 14 shows a binding result of an antibody and a tumor cell line detected by FACs in an embodiment of the present disclosure.

FIG. 15 shows a competitive binding result of the humanized BCMA antibody and BCMA ligand APRIL in an embodiment of the present disclosure.

FIG. 16 shows a result of detecting that the humanized BCMA antibody may specifically bind to a BCMA-positive cell by a flow cytometry in an embodiment of the present disclosure.

FIG. 17 is a structure schematic diagram of PCDHF-42 in an embodiment of the present disclosure.

FIG. 18 is a structure schematic diagram of PCDHF-73 in an embodiment of the present disclosure.

FIG. 19 is a structure schematic diagram of PCDHF-74 in an embodiment of the present disclosure.

FIG. 20 shows a chimeric antigen receptor (CAR) positive rate CAR+ of the CART cell in an embodiment of the present disclosure.

FIG. 21 shows a detection result of target cell apoptosis after CART and the target cell are co-cultured for 6 h in an embodiment of the present disclosure.

FIG. 22 shows a detection result of IL-2 after the CART cell and the target cell are co-cultured for 6 h in an embodiment of the present disclosure.

FIG. 23 shows a detection result of IFNγ after the CART cell and the target cell are co-cultured for 6 h in an embodiment of the present disclosure.

### DETAILED DESCRIPTION OF THE EMBODIMENTS

References of embodiments of the present disclosure are provided now in detail, and one or more examples of which are described below. Each example is provided as illustration and non-limitation to the present disclosure. In fact, it is apparent to those skilled in the art that various modifications and variations may be made in the present disclosure without departing from the scope or spirit of the present disclosure. For example, features illustrated or described as a part of one embodiment may be used in another embodiment, to produce a still further embodiment.

Therefore, it is intended that the present disclosure covers such modifications and variations falling within the scopes of the appended claims and equivalents thereof. Other objects, features and aspects of the present disclosure are disclosed in or are apparent from the following detailed descriptions. It should be understood by those of ordinary skill in the art that this discussion is the description of exemplary embodiments only, and is not intended to limit the broader aspects of the present disclosure.

The present disclosure relates to an antibody, and it is capable of specifically binding to BCMA, and selected from a) and/or b):

a) including heavy chain complementarity determining regions CDR-VH1, CDR-VH2, and CDR-VH3 of which amino acid sequences are as shown in SEQ ID NO: 1˜3, and light chain complementarity determining regions CDR-VL1, CDR-VL2, and CDR-VL3 of which amino acid sequences as shown in SEQ ID NO: 4˜6; and
b) including heavy chain complementarity determining regions CDR-VH1, CDR-VH2, and CDR-VH3 of which amino acid sequences are as shown in SEQ ID NO: 19˜21, and light chain complementarity determining regions CDR-VL1, CDR-VL2, and CDR-VL3 of which amino acid sequences as shown in SEQ ID NO: 22˜24.

In the present disclosure, “antibody” generally refers to all proteins/protein fragments containing a CDR region, especially full-length antibodies or functional antibody fragments. A term “full-length antibody” includes a polyclonal antibody and a monoclonal antibody, and a term “functional antibody fragment” is a substance containing a part or all of antibody CDRs, and it lacks at least some of amino acids present in a full-length chain but is still capable of specifically binding to an antigen. Such fragments are biologically-active because it binds to a target antigen and may competitively bind to a given epitope with other antigen-binding molecules (including a complete antibody). In some embodiments, the fragment is a fragment that has a function to block the binding of BCMA to its ligand APRIL. In some embodiments, the fragment may block or reduce the activity of BCMA. In one aspect, such fragments may contain a single heavy chain and a single light chain, or portions thereof. The fragment may be generated by a recombinant nucleic acid technology, or may be generated by enzymatic cleavage or chemical cleavage of the antigen-binding molecules (including the complete antibody).

Variants of the antibody are also within the scope of the present disclosure, for example, each of variable light chains and/or variable heavy chains having at least 70%˜80%, 80%˜85%, 85%˜90%, 90%˜95%, 95%˜97%, 97%˜99% or greater than 99% of the identity with the amino acid sequences of the sequences described herein. In some cases, the variants of the antibody at least include the above 6 CDRs; in some cases, the variants of the antibody at least include one heavy chain and one light chain, and in other cases, the variant form contains two identical light chains and two identical heavy chains (or subportions thereof). In some cases, the variants retain the ability to block the binding of BCMA to its ligand APRIL. Those skilled in the art are able to determine the suitable variants of the antigen-binding molecules as elucidated herein by using well-known technologies. In certain implementation schemes, those skilled in the art may identify suitable regions of the molecule that may be changed by targeting regions that are believed to be unimportant for the activity without destructing the activity.

The antibody provided by the present disclosure may specifically bind to the extracellular fragment of BCMA, and has the excellent specificity (it basically does not bind to other antigens on the cell membrane surface). In particular, an important advantage of the antibody is that it has the activity of blocking the binding of BCMA to its ligand APRIL, and thus is preferably used as an antibody drug.

In other implementation schemes, in view of the fact that the present disclosure verifies the affinity of the antibody to BCMA already, those skilled in the art may also judge that it may also be used as a diagnostic or verification tool without creative work. The antibody may be used to analyze the amount of BCMA present in a sample and/or a subject. In some implementation schemes, the diagnostic antibody is not an antibody for blocking the binding function of BCMA to its ligand APRIL. In some implementation schemes, the antibody disclosed herein may be used or provided in assay kits and/or methods for detection of BCMA in mammalian, especially human tissues or cells, to screen/diagnose diseases or disorders associated with changes in BCMA levels. The kit may contain an antigen-binding molecule that binds to BCMA, along with a component for indicating binding of the antigen-binding molecule to BCMA (if present) and optionally BCMA protein levels.

As used herein, “framework region” or “FR” regions mean regions of an antibody variable domain excluding those regions defined as CDRs. Each antibody variable domain framework may be further subdivided into adjacent regions (FR1, FR2, FR3, and FR4) separated by CDRs.

In general, the variable regions VL/VH of the heavy chain and the light chain may be obtained by linking the following numbered CDRs and FRs in the following combination: FR1-CDR1-FR2-CDR2-FR3-CDR3-FR4.

As used herein, a term “isolated” associated with a polypeptide or a nucleic acid means that the polypeptide or the nucleic acid is not in its natural medium or natural form. Thus, the term “isolated” includes a polypeptide or a nucleic acid removed from its original environment, for example, if it occurs in the nature, from its natural environment.

In some embodiments, the antibody a includes heavy chain framework regions FR-H1, FR-H2, FR-H3 and FR-H4 of which sequences are as shown in SEQ ID NO: 7˜10, SEQ ID NO: 41˜44 or SEQ ID NO: 45˜48; and/or light chain framework regions FR-L1, FR-L2, FR-L3 and FR-L4 of which sequences are as shown in SEQ ID NO: 11˜14, SEQ ID NO: 49˜52, SEQ ID NO: 53˜56 or SEQ ID NO: 57˜60 sequentially.

In some embodiments, the antibody b includes heavy chain framework regions FR-H1, FR-H2, FR-H3 and FR-H4 of which sequences are as shown in SEQ ID NO: 25˜28 sequentially; and/or light chain framework regions FR-L1, FR-L2, FR-L3 and FR-L4 of which sequences are as shown in SEQ ID NO: 29˜32 sequentially.

In some embodiments, the antibody is one of F(ab′)2, Fab, Fv, scFv, and a bispecific antibody. In further implementation schemes, the antibody is a single chain variable fragment (scFv).

In some embodiments, the antibody has a constant region, and a constant region sequence is selected from a sequence of any one of constant regions of IgG1, IgG2, IgG3, IgG4, IgA, IgM, IgE, and IgD.

In some embodiments, the species source of the constant region is independently selected from bovine, equine, dairy cow, pig, sheep, goat, rat, mouse, dog, cat, rabbit, camel, donkey, deer, mink, chicken, duck, goose, turkey, cockfight or human.

The present disclosure further relates to a chimeric antigen receptor, and it contains the antibody as described above.

It should be understood that the antibody type contained in one preferred orientation of the chimeric antigen receptor according to the present disclosure is sc-Fv.

In some embodiments, the chimeric antigen receptor further contains one or more elements selected from a group consisting of the following components: a leader peptide, a linker sequence, a transmembrane domain, a costimulatory domain, and a signal conduction domain.

In some embodiments, the leader peptide is selected from a CD8 leader chimeric receptor signal peptide.

In some embodiments, the leader peptide is selected from a linker sequence selected from a hinge region of CD8.

In some embodiments, the transmembrane domain is selected from a transmembrane region of CD8.

In some embodiments, the costimulatory domain is a signal transmission region of the following items: CD28, CD28T, OX-40, 4-1BB/CD137, CD2, CD7, CD27, CD30, CD40, programmed death-1 (PD-1), inducible T cell costimulatory factor (ICOS), lymphocyte function-associated antigen-1 (LFA-1, CDI-Ia/CD18), CD3γ, CD3δ, CD3ε, CD247, CD276 (B7-H3), LIGHT, (TNFSF14), NKG2C, Igα (CD79a), DAP-10, Fcγ receptor, MHC class-1 molecule, TNF receptor protein, immunoglobulin protein, cytokine receptor, integrin, signal lymphocyte activation molecule (SLAM protein), activated NK cell receptor, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8α, CD8β, IL-2Rβ, IL-2Rγ, IL-7Rα, ITGA4, VLA1, CD49a, ITGA4, IA4, CD49D, ITGA6, VLA-6, CD49f, ITGAD, CDIId, ITGAE, CD103, ITGAL, CDI Ia, LFA-1, ITGAM, CDIIb, ITGAX, CDI Ic, ITGBI, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, LyI08), SLAM (SLAMF1, CD150, IPO-3), BLAME (SLAMF8), SELPLG (CD162), LTBR, LAT, GADS, SLP-76, PAGICbp, CD19a, a ligand that specifically binds to CD83 or any combinations thereof.

In some embodiments, the signal conduction domain is selected from any one of PKCθ, FcεRIγ, ZAP70, CD3ζ, or any combinations thereof.

In some embodiments, the functional fragment includes a CD8 leader chimeric receptor signal peptide, a hinge region of CD8, a transmembrane region of CD8, CD137 and CD3ζ.

The present disclosure further relates to an isolated nucleic acid, and it encodes the antibody as described above, or the chimeric antigen receptor as described above.

The present disclosure further relates to a vector, and it contains the isolated nucleic acid as described above.

A term “vector” refers to a nucleic acid delivery tool into which a polynucleotide may be inserted. While the vector may express a protein encoded by the inserted polynucleotide, the vector is called an expression vector. The vector may be introduced into a host cell by transformation, transduction or transfection, so that a genetic material element carried by it may be expressed in the host cell. The vector is well-known to those skilled in the art, and includes, but is not limited to: a plasmid; a phagemid; a cosmid; an artificial chromosome, such as a yeast artificial chromosome (YAC), a bacterial artificial chromosome (BAC) or a P1-derived artificial chromosome (PAC); and a phage such as a λ phage or a M13 phage and an animal virus. The animal virus that may be used as the vector includes, but is not limited to, a retrovirus (including a lentivirus), an adenovirus, an adeno-associated virus, a herpesvirus (such as a herpes simplex virus), a poxvirus, a baculovirus, a papillomavirus, a papovavirus (such as SV40). In some embodiments, the vector of the present disclosure contains a regulatory element commonly used in genetic engineering, for example, an enhancer, a promoter, an internal ribosome entry site (IRES), and other expression control elements (such as a transcription termination signal, or a polyadenylation signal and a polymeric U sequence).

In some embodiments, the vector of the present disclosure may further contain fragments of a gene used for screening (for example, an antibiotic resistance gene), a nucleic acid used to generate a fluorescent protein and the like. The fluorescent protein may be selected from a green fluorescent protein, a blue fluorescent protein, a yellow fluorescent protein, an orange fluorescent protein or a red fluorescent protein.

In particular, while the nucleic acid encoding the chimeric antigen receptor as described above is contained in the vector, the vector is preferably a retroviral vector, more preferably a lentiviral vector.

In a specific embodiment, a nucleotide sequence of the lentiviral vector is shown in SEQ ID NO: 18.

The present disclosure further relates to a host cell, and it contains the vector as described above.

A term “host cell” refers to a cell that may be used to introduce the vector, and it includes, but is not limited to, a prokaryotic cell such as Escherichia Coli or Bacillus subtilis, a fungal cell such as a yeast cell or aspergillus, an insect cell such as an S2 drosophila cell or Sf9, or an animal cell such as a fibroblast, a CHO cell, a COS cell, a NSO cell, a HeLa cell, a BHK cell, a HEK 293 cell or a human cell. The host cell is preferably a eukaryotic cell, more preferably a mammalian cell.

The present disclosure further relates to an immune cell, and it contains the chimeric antigen receptor as described above.

In some embodiments, the immune cell is a T cell, a tumor infiltrating lymphocyte (TIL cell), a NK cell, a dendritic cell, or a NK-T cell.

In some embodiments, the immune cell is an autologous T cell or an allogeneic T cell.

In some embodiments, the immune cell is obtained or prepared from peripheral blood.

In some embodiments, the immune cell is obtained or prepared from a peripheral blood mononuclear cell (PBMC).

In some embodiments, the immune cell is obtained or prepared from bone marrow.

In some embodiments, the immune cell is obtained or prepared from umbilical cord blood.

In some embodiments, the immune cell is the human cell.

The present disclosure further relates to a pharmaceutical composition, and it includes the antibody as described above or the immune cell as described above, and one or more of a pharmaceutically acceptable excipient, diluent or carrier.

A term “pharmaceutically acceptable excipient, diluent or carrier” refers to an excipient, a diluent or a carrier that is pharmacologically and/or physiologically compatible with a subject and an active ingredient, and it is well-known in the fields of, including but not limited to: a pH adjuster, a surfactant, an adjuvant, and an ionic strength enhancer. For example, the pH adjuster includes but is not limited to a phosphate buffer; the surfactant includes but is not limited to a cationic, anionic or nonionic surfactant such as Tween-80; and the ionic strength enhancer includes but is not limited to a sodium chloride.

The pharmaceutical composition may be used for BCMA-related diseases, especially multiple myeloma. Thus, in particular, the present disclosure further relates to a method for treating multiple myeloma in a subject in need thereof, and the method includes:

a) providing the pharmaceutical composition; and

b) administering a therapeutically effective amount of the pharmaceutical composition to the subject.

A term “effective amount” refers to an amount sufficient to obtain, or at least partially obtain, a desired effect. For the desired effect, for example, the prevention or treatment of the multiple myeloma, the effective amount is generally an amount sufficient to prevent, stop, or delay the occurrence of the disease. Determining such effective amount is well within the ability of those skilled in the art. For example, the effective amount for therapeutic use may depend on the severity of the disease to be treated, the general state of an own immune system of the subject, the general conditions of the subject such as an age, a weight and a sex, the administration mode of a drug, and other treatments administered concurrently and the like.

In some embodiments, the method of administration may adopt administration by injection and the like.

In the present disclosure, terms “subject”, “patient” and the like may be commonly used as needed. The subject may be a mammal, preferably a human.

The implementation schemes of the present disclosure are described in detail below with reference to the embodiments.

### Embodiment 1: Acquisition of Antibody Targeting BCMA

A conjugate of a human BCMA extracellular fragment and a mouse antibody Fc fragment (Fragment crystallizable) (hereinafter referred to as hBCMA-mFc, ACRO, BCA-H5253) is injected intraperitoneally into BALB/c mice (Guangdong Medical Laboratory Animal Center), each is injected once a week, and each time is 100 μg/200 μl/mouse, after 3 weeks of immunization, tail blood of mice is collected every week to detect the expression of the BCMA antibody in serum; the mice with the high BCMA antibody expression in the serum are selected to acquire spleen cells, the spleen cells are fused with tumor cells (SP20, ATCC HB-12546) to form a fusion, and the fusion expressing the BCMA antibody in a culture supernatant is selected for monocloning after being cultured for 10˜14 days; a monoclonal hybridoma cell line expressing the BCMA antibody is selected for amplification culture, after being cultured for 7˜10 days, cell culture solution is collected and purified to obtain the BCMA antibodies, and after a large number of the obtained antibodies are screened, two candidate antibodies (5E2 and 5F4) are obtained, 5E2 is sequenced, an amino acid sequence of a heavy chain variable region VH is shown in SEQ ID NO: 15, and an amino acid sequence of a light chain variable region VL is shown in sequence SEQ ID NO: 16; and 5F4 is sequenced, an amino acid sequence of a heavy chain variable region VH is shown in SEQ ID NO: 33, andanamino acid sequence of a light chain variable region VL is shown in sequence SEQ ID NO: 34.

### Embodiment 2: Screening of BCMA Antibodies

1) BCMA antibody (5E2, 5F4) affinity detection:

The affinity of different antibodies for BCMA is detected by three modes of ELISA, Fortebio and FACs, and a specific method is as follows.

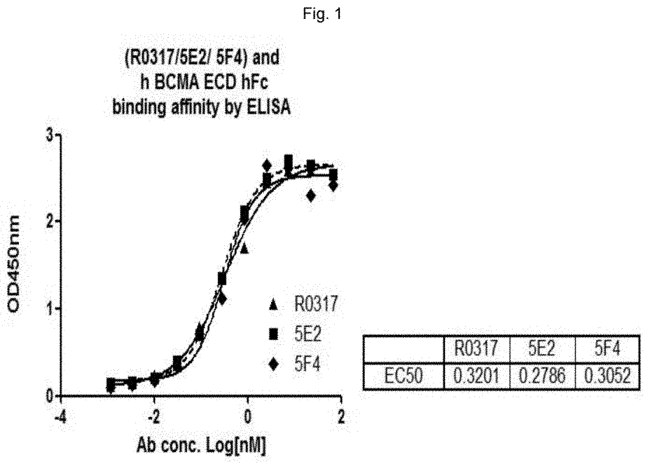

Antibody affinity ELISA detection: hBCMA ECD hFc, ACRO, BC7-H5254 is coated in a 96-well enzyme-linked coating plate at a concentration of 2 μg/ml and 100 μl/well, the antibodies diluted in a 3-fold gradient (the former concentration is 3 times greater than the latter concentration, diluted with 1% bull serum albumin (BSA)) bind to the antigens, and a EC₅₀ value of each antibody is detected (the specific operation steps are general ELISA operation steps). R0317 is a C11D5.3BCMA mouse monoclonal antibody, and its specific sequence and information may be found publicly. The antibody to be detected is synthesized by Shenzhen Feipeng Biological Therapy Co., Ltd. Results, shown in FIG. 1, show that the BCMA antibodies 5E2 and 5F4 have EC₅₀ at the same level as R0317.

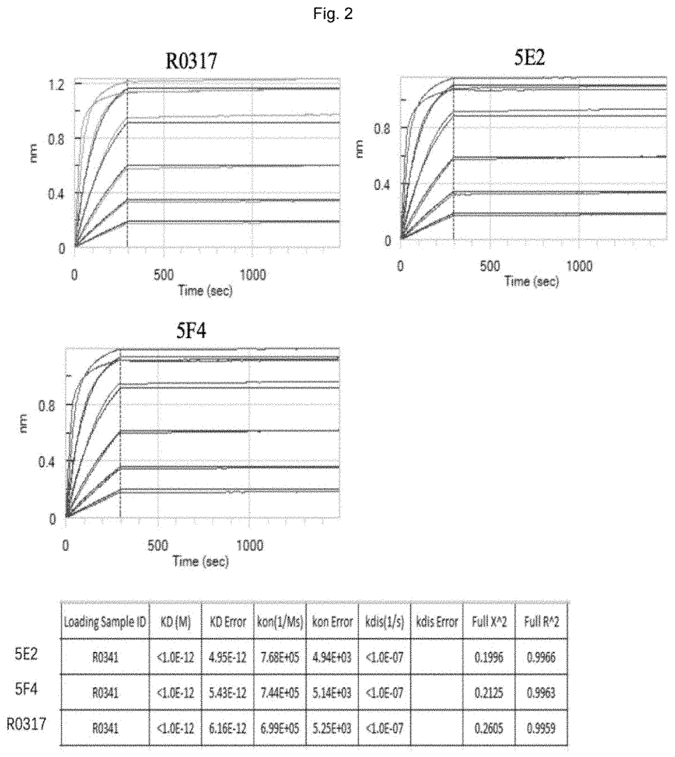

The antibody affinity is detected by Fortebio, ProG biosensor (Pall ForteBIO, 18-1502) is used, firstly a buffer (phosphate buffered saline (PBS)+0.02% Tween20) is loaded, then 5 μg/mL of h BCMA ECD hFc (R0341) is added, and then three antibodies are added respectively, KD, Kon and Kdis of the 3 antibodies are detected. The specific operation steps are routine operations using a Fortebio instrument (forteBio, Serial NO: FB-40476). Detection results are shown in FIG. 2, and it is indicated that 5E2 and 5F4 have the stronger affinity with the BCMA antigen and are at the same level as R0317.

Binding of Antibody to Tumor Cell Line Detected by FACs:

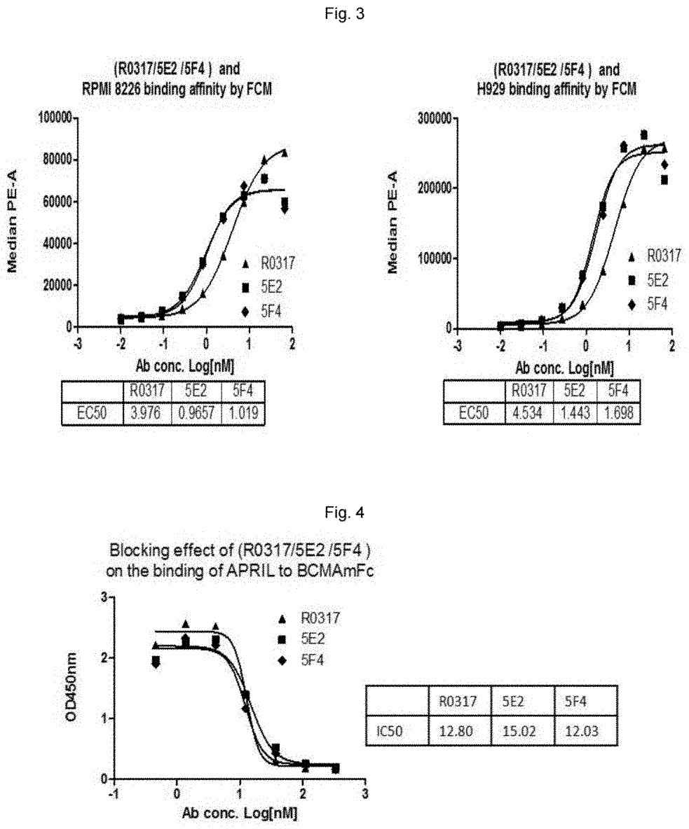

A RPMI8226 cell (ATCC® CRM-CCL-155™) is a human multiple myeloma peripheral blood B lymphocyte, and a H929 cell (ATCC® CRL-9068™) is a human multiple myeloma bone marrow B lymphocyte. A specific detection method is as follows: cells are harvested, washed with PBS once, and then resuspend with PBS according to 2E+5 cells/200 μl. The three antibodies are diluted in a gradient (the initial concentration of the antibody is 10 μg/ml, it is diluted with 1% BSA in 3-fold, and there is a total of 9 gradients), and then incubated with the cells for 30 min at 4° C. After that, it is incubated with a PE-labeled anti-mouse IgG secondary antibody (Biolegend, B288920), washed twice, and detected by a Beckman Coulter (model number: CytoFLEX) flow cytometry. As shown in FIG. 3, 5E2, and 5F4 have concentration gradient-dependent binding to RPMI8226 and H929 cells and EC₅₀ is significantly lower than that of R0317, it is indicated that the affinity of 5E2, and 5F4 with RPMI8226 and H929 cells is better than that of R0317.

2) Competitive Binding Experiment of BCMA Antibody (5E2, 5F4) and BCMA Ligand APRIL:

ACRO recombinant human APRIL Ala-Leu 250+N-terminal linked human IgG1 Fc (article number: APL-H5267) is purchased. hBCMA-mFc is coated onan ELISA plate at 4 μg/ml, and coated overnight at 4° C. After being washed once with PBS, it is blocked with 1% BSA for 1 h, and then washed once again with PBS. The antibodies 5E2 and 5F4 are diluted in a gradient, the initial concentration is 200 μg/ml, it is diluted with 1% BSA in a 3-fold gradient, and there is a total of 7 gradients, a diluent is 4 μg/ml of hAPRILhFc (this concentration is between EC₅₀ of APRIL binding to hBCMA-mFc and saturation), it is incubated at 37° C. for 30 min, and washed for 5 times, a HRP-labeled goat anti-human Fc antibody (Goat Anti-Human IgG Antibody, Fc, HRP conjugate Sigma, AP113P) diluted in 1:1500 is added, incubated at 37° C. for 30 min, and washed for 5 times, the color is developed with tetramethylbenzidine (TMB), and it is read by a multi-function microplate reader after termination. Results are shown in FIG. 4. IC₅₀ of 5E2 and 5F4 for blocking APRIL is comparable to that of R0317, and the blocking effects of 5E2 and 5F4 on APRIL and hBCMA-mFcare apparent and concentration-gradient-dependent.

3) BCMA Antibody (5E2, 5F4) Specificity

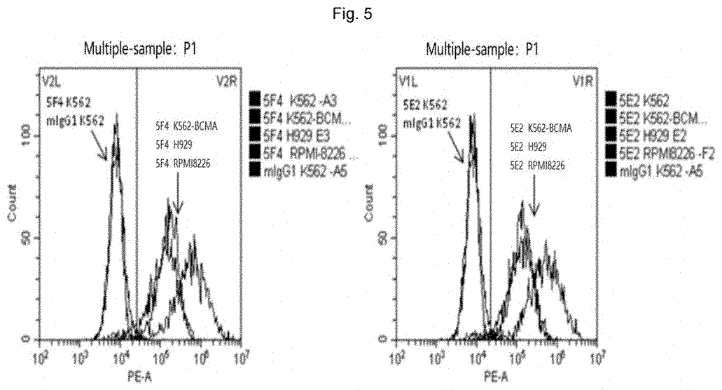

The specificity of BCMA antibody of the present disclosure is detected by FACs. A human BCMA full-length gene is synthesized in vitro, and a restriction enzyme cutting site is introduced, the synthesized human BCMA full-length gene is inserted into a lentiviral packaging plasmid vector (PCDHF) by double enzyme digestion, and lentiviral packaging (the specific operation steps are generally 4 plasmid lentiviral packaging steps, please refer to a section about lentiviral packaging on an official website of Jikai Gene in detail) is performed. The prepared lentivirus is infected with a K562 cell (ATCC, article number: CCL-243) to construct a K562-BCMA cell, and the cells are single-clone-picked and identified, to obtain a stable cell line K562-BCMA stably expressing BCMA. The 5E2 and 5F4 antibodies are respectively incubated with K562, H929, RPMI8226 and K562-BCMA cells for 30 min at 4° C., after that, it is washed twice with PBS, then a secondary antibody PE goat anti-mouse IgG1 (Biolegend, B288920) is added, and incubated at 4° C. for 30 min, after that, it is washed twice with PBS. Then, the detection is performed by using a Beckman Coulter flow cytometry. Detection results are shown in FIG. 5 below, and 5E2 and 5F4 may specifically bind to the cells of which the BCMA expression is positive.

### Embodiment 3: Construction of BCMA CART Cell and In Vitro Functional Verification

1) Acquisition of 5E2 and 5F4 Antibody Sequences:

Hybridoma cell lines expressing 5E2 and 5F4 are recovered, and after 72 hours of normal culture, cells are lysed to extract a ribonucleic acid (RNA) (extraction kit: TOYOBO LIFE SCIENCE, article number: 836700, and the extraction steps are in accordance with instructions). The extracted RNA is reverse-transcribed to obtain cDNA (reverse transcription kit: TOYOBO LIFE SCIENCE, and article number: 11141ES10), and the obtained cDNA is PCR-amplified (a specific primer for a mouse IgG1 sequence is used) to obtain an antibody sequence and it is sequenced, and antibody sequencing VH and VL refer to VH, VL sequences of labeled 5E2 and 5F4 in Embodiment 1. Since the affinity, specificity and functional properties of the two antibodies are similar, 5E2 is used as an example to verify a CART function.

2) Lentivirus Packaging:

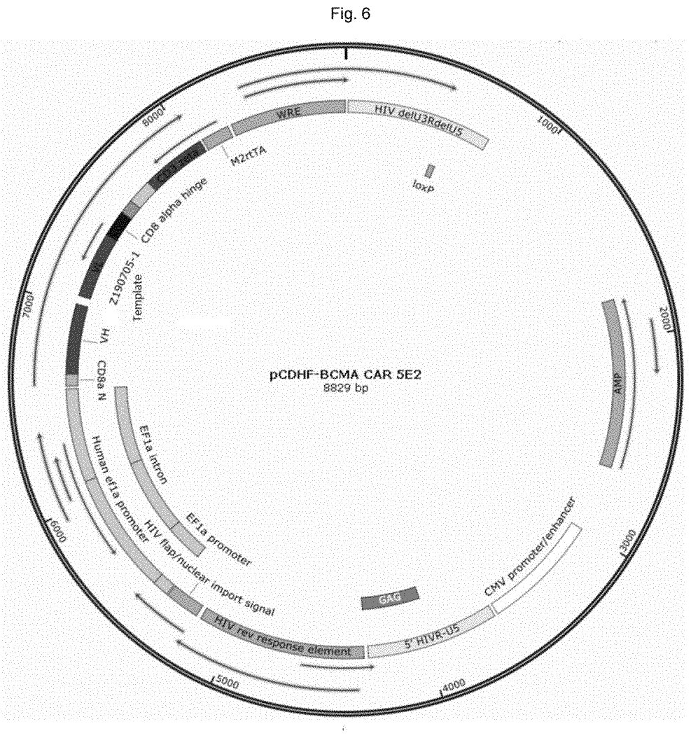

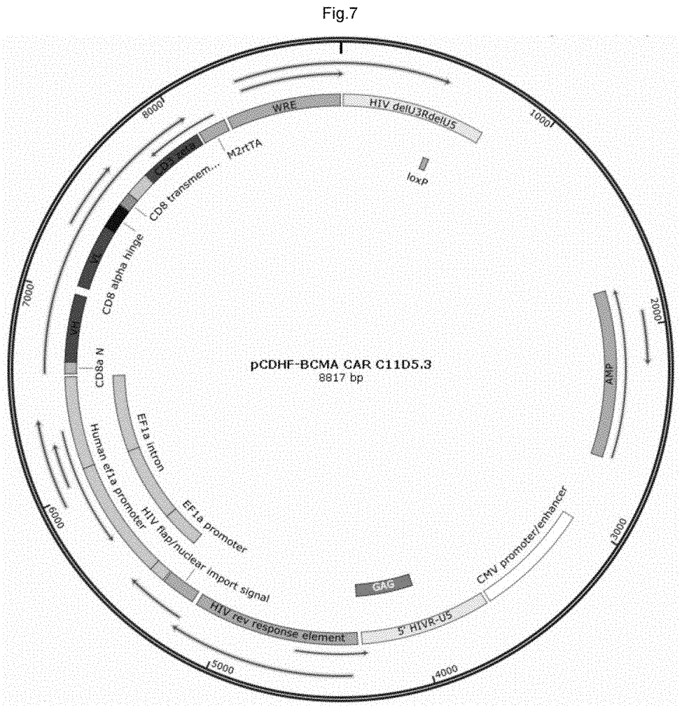

A scFv sequence of the 5E2 antibody (it is obtained by connecting a C-terminal of VH to an N-terminal of VL by a linker peptide, and a nucleotide sequence of the linker peptide is shown in SEQ ID NO: 35) and a scFv sequence of C11 D5.3 (SEQ ID NO: 17) are respectively constructed on a lentiviral vector (PCDHF) to obtain a CAR plasmid, and structure schematic diagrams of the C11D5.3CAR plasmid and the 5E2CAR plasmid are shown in FIG. 6 and FIG. 7. A 293T cell (ATCC® CRL-3216™) is used to package the lentivirus, a packaging system and packaging steps are as follows:

a. 293T cells 5E6 are inoculated in a 10 cm cell culture dish, 10 mL of a DMEM medium containing 10% FBS (DMEM Gibco, 11995040-1L; FBS Gibco, 10091-148) is added, and it is cultured under conditions of 5% CO₂ and 37° C. in an incubator for 48 h.

b. Lentiviral Packaging System

| | Lentivirus name | |
|---|---|---|
| Plasmid name | 5E2 | C11D5.3 |
| PCDHD(PMD2.G*¹) | 6 μg | 6 μg |
| PCDHM(pMDLg/pRRE*²) | 6 μg | 6 μg |
| PCDHN(pRSV-Rev*³) | 6 μg | 6 μg |
| PCDHF-5E2 | 12 μg | |
| PCDHF-C11D5.3 | | 12 μg |
| PEI(Polysciences, 636951) | 60 μg | 60 μg |
| OPTI-MEMI(Gibco, 31985070) | 3 mL | 3 mL |

Herein, “*¹, *², *³” are lentiviral packaging plasmid PMD2.G, pMDLg/pRRE, and pRSV-Rev sequences, and may be obtained by a public way (website http://www.miaolingbio.com/).

c. 48 hours after packaging, a cell supernatant is collected, and the lentivirus titer is detected after ultracentrifugation at 25,000 rpm. A detection method is as follows: 293T cells are infected with the collected lentivirus stock solution under a condition of the same gradient volume, and after 48 h, the percentage of GFP positive rate of the 293T cell is detected by a flow cytometry, according to a calculation formula: stock solution titer (TU/mL)=1.5×10E+05×293T cell GFP positive rate percentage/lentivirus stock solution volume μl×1000, the lentivirus stock solution titer is calculated.

3) CART Cell Preparation

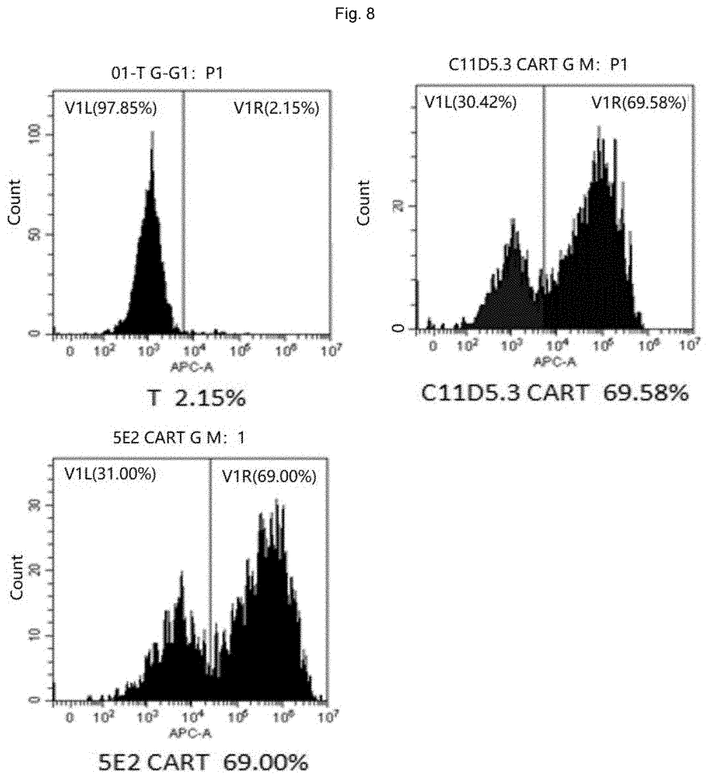

Ficoll lymphatic separation solution (Dakwin, AS1114546) is used to separate PBMC cells from blood (50 mL of volunteer blood donated by a Feipeng Biotech employee No. 0038), and T cells are separated and obtained by a magnetic bead positive selection method coupled with a CD3/CD28 antibody. T cells are infected at MOI=5:1 to prepare CART cells, and the CAR positive rate of the CART cells is detected by the flow cytometry with a secondary antibody APC goat anti-mouse IgG (H+L) (Jackson, 115-136-146) after the CART cells are cultured for 7 days, as shown in FIG. 8.

4) In Vitro Functional Evaluation of CART Cell

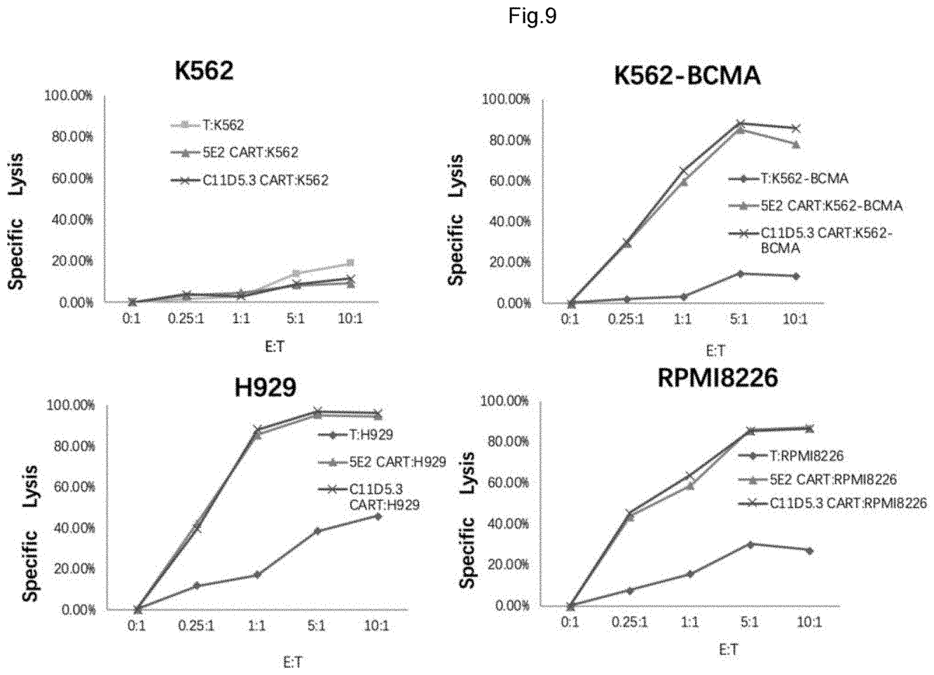

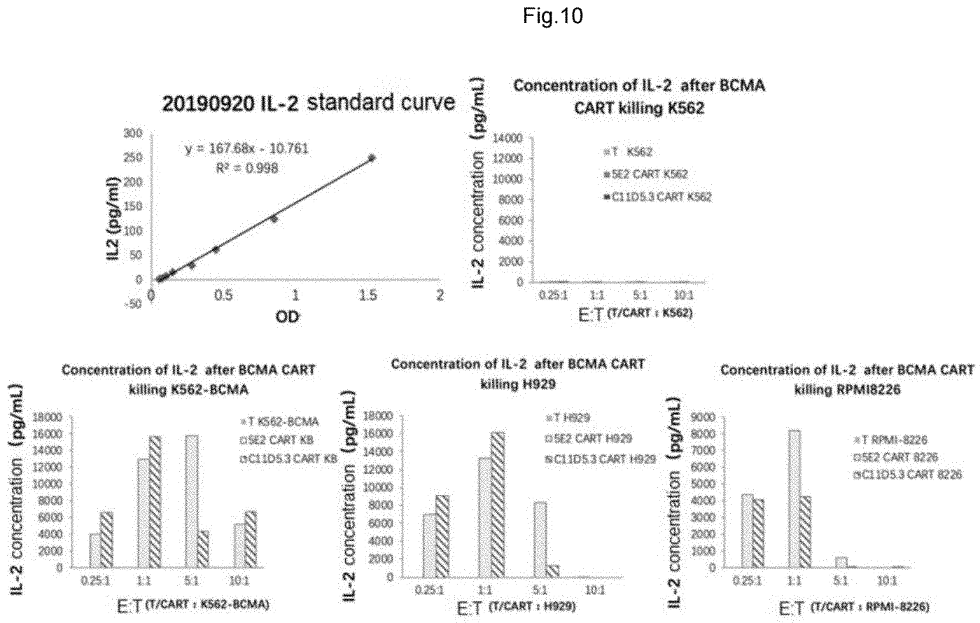

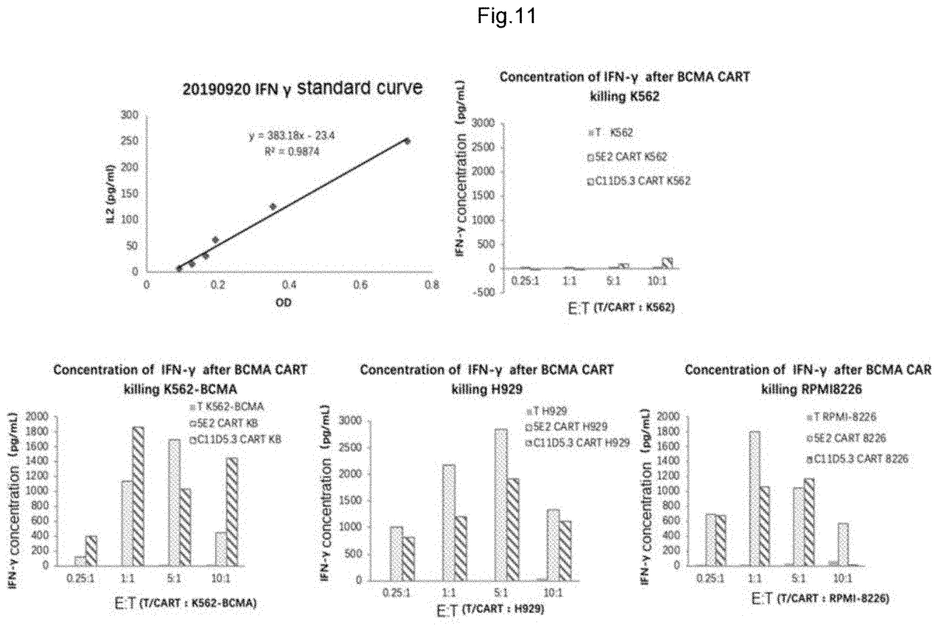

4 target cells K562, K562-BCMA, H929 and RPMI8226 are respectively taken, each is 2×10E+06 cells, and firstly CytoCalcein™ Violet 550 is used to stain the target cells at 1×10E+05 cells/100 μl/well. Effector cells (CAR+CART, T cells are used as the control) and the above target cells are added to a 96-well plate at the ratio of 0.25:1, 1:1, 5:1 and 10:1 respectively and mixed uniformly, and the final volume is 200 μl. After 8 h of culture, the cells are mixed uniformly and centrifuged. A supernatant is detected by a Human IL-2 ELISA detection kit (invitrogen, REF 88-7025-88) and a Human IFN gamma ELISA kit (invitrogen, REF 88-7316-88) is used to detect the IL-2 and IFN-γ concentrations in each well, and a precipitation portion is resuspended with 100 μl of Annexin V Binding Buffer (Biolegend, B274722), it is centrifuged at 300 g for 5 min, after that, 2.0 μl of APC-Annexin V (Biolegend, Cat 640920) and 1.2 μl of PI dye (Biolegend, Cat 421301) are added, it is incubated in the dark for 15 min. 100 μl of Annexin V Binding Buffer is added to resuspend, after that, the Beckman Coulter flow cytometry is used to detect the apoptosis ratio of each target cell (results are shown in FIG. 9), and the concentration of IL-2 and IFN-γ in the supernatant of each well are detected by ELISA (results are shown in FIG. 10 and FIG. 11). Herein, K562 is a BCMA-negative cell, and K562-BCMA, H929, and RPMI8226 are all BCMA-positive cells. Results show that 5E2CART and C11 D5.3CART have the stronger specific killing effect on the BCMA-positive target cells, and 2 types of the CART cells have basically the same ability to kill the positive target cells, but have almost no killing effect on the BCMA-negative cells.

### Embodiment 4: Acquisition of Humanized Antibody Targeting BCMA

Four antibody sequences including two heavy chain variable regions (SEQ ID NO:36-SEQ ID NO:37) and three light chain variable regions (EQ ID NO:38-SEQ ID NO:40) were obtained after humanization of the murine 5E2 antibody.

### Embodiment 5: Evaluation of BCMA Humanized Antibody

1) Humanized BCMA Antibody Affinity Detection

The binding affinity of 4 BCMA humanized antibodies (hu VH1-VL1; hu VH2-VL1; hu VH1-VL2; and hu VH1-VL3) withBCMA antigens are detected by ELISA, Fortebio and FACs, and a specific method is as follows.

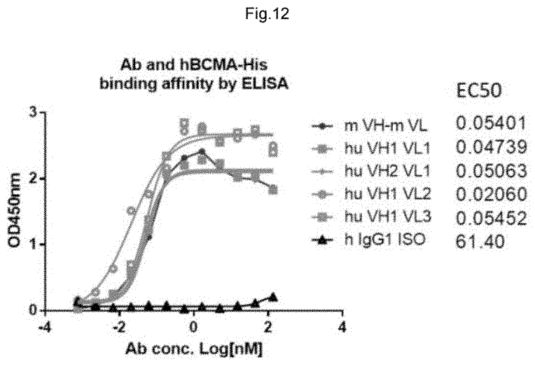

Antibody affinity ELISA detection: hu (human) BCMA ECD His, ACRO, BCA-H522y is coated on a 96-well enzyme-linked coated plate at a concentration of 2 μg/mL and 100 μL/well, and the 4 humanized antibodies are respectively prepared into an initial concentration of 20 μg/mL with 1% BSA, it is diluted in a 3-fold gradient with 1% BSA (the concentration of the former gradient antibody is 3 times greater than the concentration of the latter gradient antibody), the antibodies bind to the antigens, and EC₅₀ values of the four antibodies are detected (the specific operation steps are general ELISA operation steps). Herein, mVH-mVL is a murine BCMA antibody, and is a murine antibody before humanization. Results are shown in FIG. 12 below, and the results show that the 4 humanized BCMA antibodies and the murine BCMA antibody mVH-mVL have the same level of EC₅₀, and hu VH1-VL2 has the highest affinity.

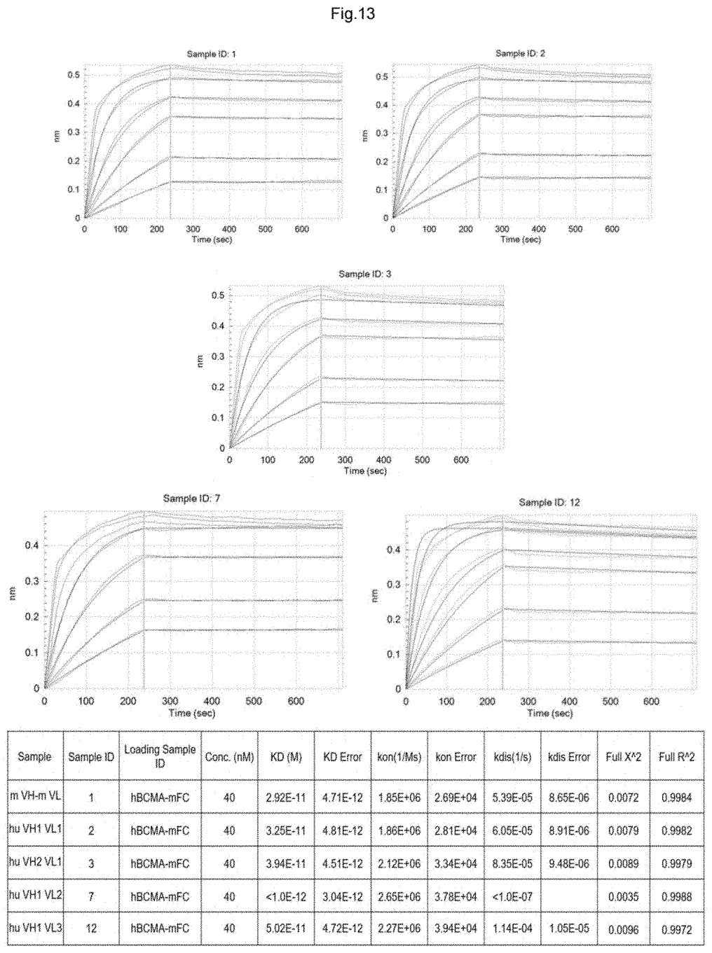

The antibody affinity is detected by Fortebio, AMC biosensor (Pall, lot: 1907292) is used, and firstly 3 μg/mL of h BCMA ECD mFc is loaded, and then it binds to four humanized antibodies respectively, to detect KD, Kon and Kdis of the 4 antibodies respectively. The specific operation steps are routine operations using a Fortebio instrument (forteBio, Serial NO: FB-40476). Detection results are shown in FIG. 13 below, and it is indicated that the four BCMA humanized antibodies have the stronger affinity to the antigens and are at the same level as the murine BCMA antibody mVH-mVL, and hu VH1-VL2 has the highest affinity.

Binding of Antibody to Tumor Cell Line Detected by FACs:

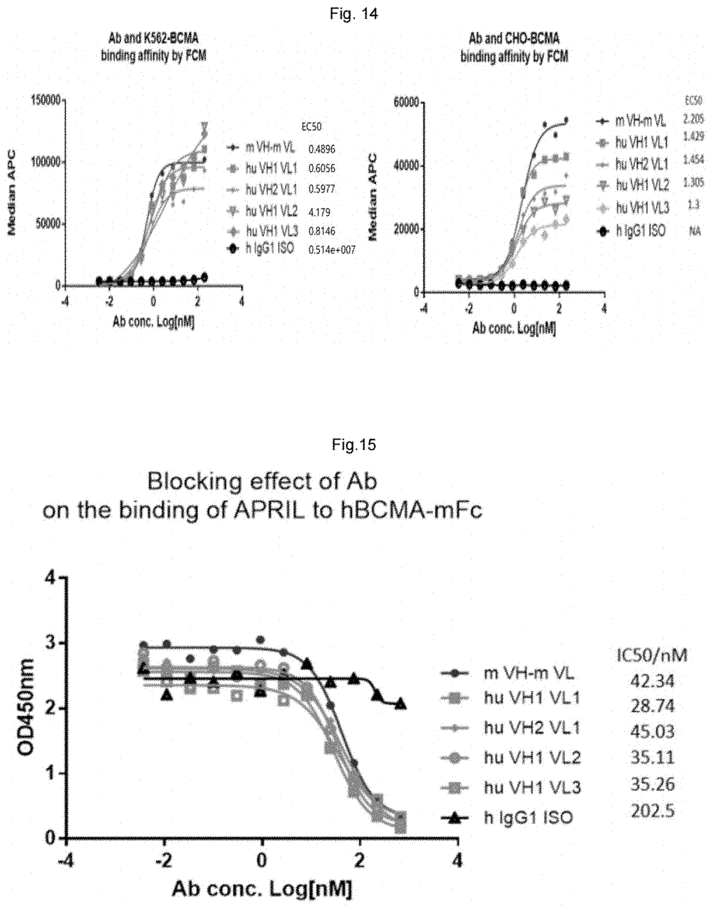

A K562 cell (ATCC® CCL-243™) is a human chronic myeloid leukemia cell, and a CHO cell (ATCC® CRL-12023™) is a Chinese hamster ovary cell. The K562 cells and the CHO cells are respectively infected with lentiviruses containing a full-length sequence of human BCMA, and after being infected, a monoclone is picked, to obtain a K562-BCMA cell line and a CHO-BCMA cell line stably expressing BCMA, and the affinity (EC50) of the four humanized antibodies to K562-BCMA and CHO-BCMA is detected respectively. A specific detection method is as follows: cells are harvested, washed once with PBS, and resuspended with PBS according to 2E+5 cells/200 μL. The four BCMA humanized antibodies are diluted in a 3-fold gradient with 1% BSA, the concentration of the former gradient antibody is 3 times greater than that of the latter gradient antibody, (the initial concentration of the antibody is 30 μg/ml, and there is a total of 11 gradients), and then it is respectively incubated with the cells at 4° C. for 30 min. After that, it is incubated with APC anti-human IgG Fc Antibody (Biolegend, 409306) for 30 min at 4° C., washed twice with 1x PBS, and detected by a Beckman Coulter (model number: CytoFLEX) flow cytometry. As shown in FIG. 14 below, 3 humanized antibodies bind to K562-BCMA cells and CHO-BCMA cells in a concentration-dependent manner and EC₅₀ is at the same level as that of the murine BCMA antibody. The affinity EC₅₀ of hu VH1VL2 to K562-BCMA is slightly weaker. However, the affinity EC₅₀ of hu VH1-VL2 and CHO-BCMA is at the same level as the other three humanized antibodies, and the expression abundance of the four humanized antibodies and the murine BCMA antibody binding to CHO-BCMA is in the order of m VH-m VL, hu VH1-VL1, hu VH2-VL1, hu VH1-VL2, and hu VH1-VL3 from high to low.

2) Competitive Binding Experiment of Four Humanized BCMA Antibodies and BCMA Ligand APRIL:

ACRO recombinant human APRIL Ala-Leu 250+N-terminal His tag (Human APRIL/TNFSF13 Protein, His Tag, article number: APL-H5244) is purchased. hBCMA-mFc is coated on an ELISA plate at 4 μg/ml, and coated overnight at 4° C. After being washed once with 1×PBS, it is blocked with 1% BSA (Sangon Biotech, A500023-0100) for 1 h, and then washed once again with 1×PBS. The four humanized antibodies are diluted in a 3-fold gradient, the concentration of the former gradient antibody is 3 times greater than that of the latter gradient antibody, the initial concentration is 100 μg/ml, there is a total of 11 gradients, a diluent is 0.2 μg/ml of h APRIL His (this concentration is between EC₅₀ of APRIL binding to hBCMA-mFc and saturation), h APRIL His is dissolved with 1% BSA, incubated at 37° C. for 30 min, and washed for 5 times with 1 xPBS, and a HRP-labeled His antibody (Anti-his tag HRP, Biolegend, 652504) diluted in 1:1500 is added, incubated at 37° C. for 30 min, and washed for 5 times, the color is developed with TMB, and it is read by a multifunctional microplate reader after termination. Results are shown in FIG. 15 below, IC50 of the 4 humanized BCMA antibodies for blocking APRIL is comparable to that of the murine BCMA antibody m VH-m VL, and the 4 humanized BCMA antibodies have the significant blocking effects on APRIL and hBCMA-mFc and concentration-gradient-dependent.

3) Specificity of Four Humanized BCMA Antibodies

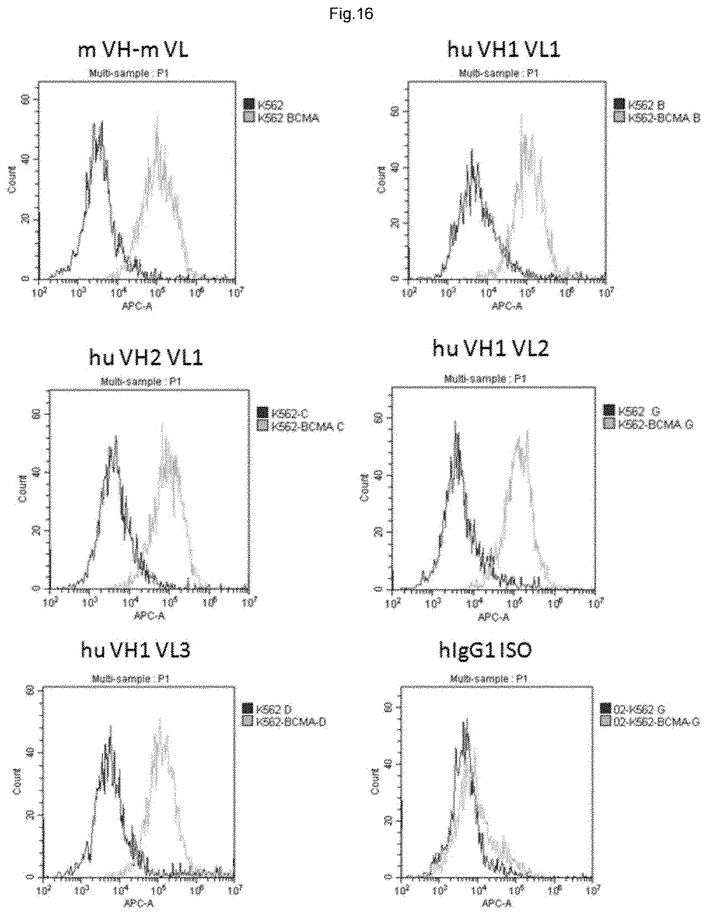

The four humanized BCMA antibodies are subjected to specific flow detection, 100 μl of the four humanized BCMA antibodies 10 μg/ml is respectively incubated with K562, K562-BCMA, CHO, CHO-BCMA and K562-BCMA cells at 4° C. for 30 min. After that, it is washed twice with 1×PBS, and then APC anti-human IgG Fc Antibody (Biolegend, 409306) is added and incubated at 4° C. for 30 min, after that, it is washed twice with 1×PBS. Then, detection is performed by using a Beckman Coulter flow cytometry. Detection results are shown in FIG. 16 below, and the 4 humanized BCMA antibodies may specifically bind to BCMA-positive cells.

### Embodiment 6: Construction of BCMA CART Cells and In Vitro Functional Verification

1) Lentiviral Packaging:

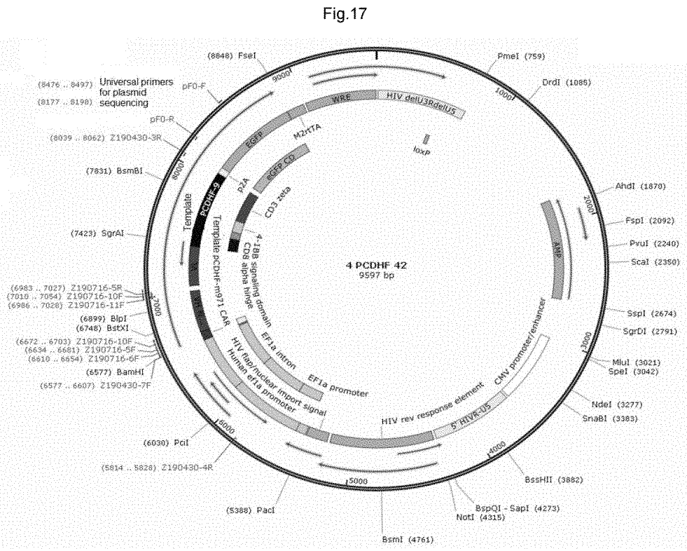

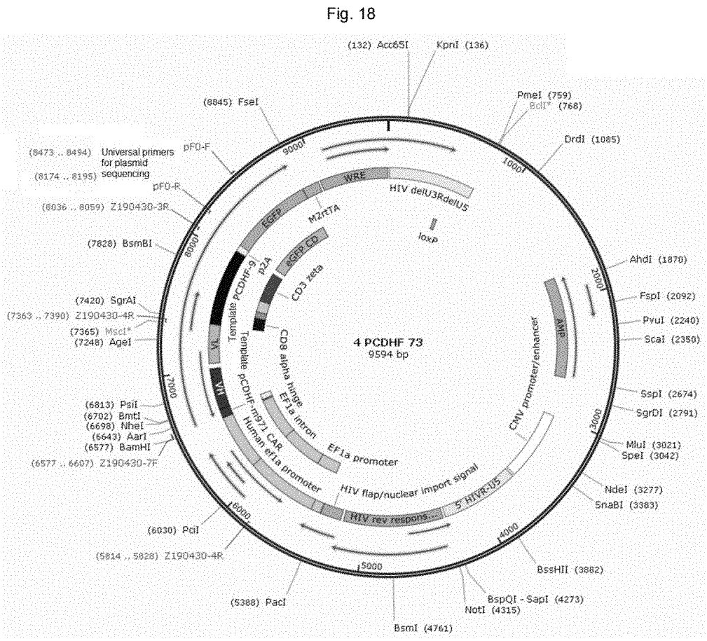

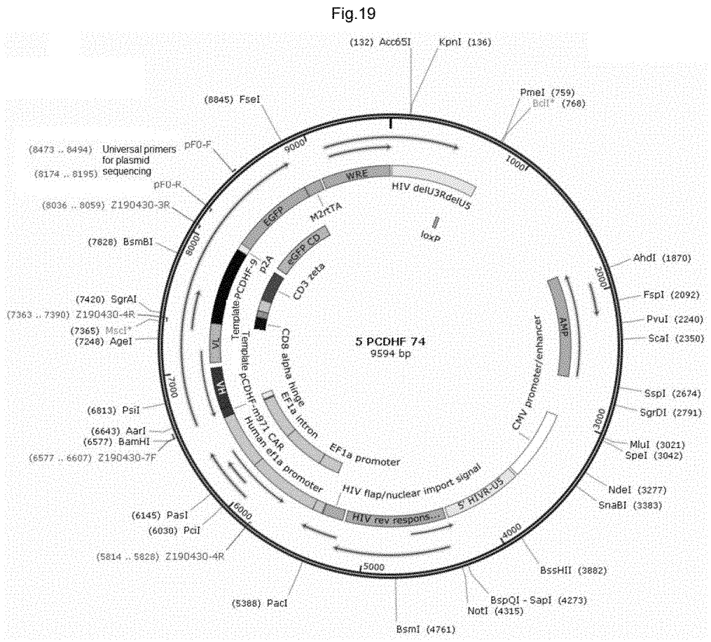

After comparison of the affinity, the blocking function and the specificity, it is preferable to construct scFv sequences of m VH-m VL, hu VH1-VL1, and hu VH2-VL1 antibodies respectively on a lentiviral vector (PCDHF, containing a GFP sequence) to obtain a CAR plasmid, the CAR plasmid obtained by the construction of the scFv sequences of m VH-m VL, hu VH1-VL1, and hu VH2-VL1 antibodies is PCDHF-42, PCDHF-73, and PCDHF-74 respectively, and structure schematic diagrams are shown in FIGS. 17, 18, and 19. 293T cells (ATCC® CRL-3216™) are used to package the lentivirus, and a packaging system and packaging steps are as follows:

a. 293T cells 5E6 are inoculated in a 10 cm cell culture dish, 10 mL of a DMEM medium containing 10% FBS (DMEM Gibco, 11995040-1L; FBS Gibco, 10091-148) is added, and it is cultured under conditions of 5% CO₂ and 37° C. in an incubator for 24 h.

b. Lentiviral Packaging System

| | Lentivirus name | | |
|---|---|---|---|
| Plasmid name | PCDHF-42 | PCDHF-73 | PCDHF-74 |
| PCDHD(PMD2.G) | 6 μg | 6 μg | 6 μg |
| PCDHM(pMDLg/pRRE) | 6 μg | 6 μg | 6 μg |
| PCDHN(pRSV-Rev) | 6 μg | 6 μg | 6 μg |
| PCDHF-42 | 12 μg | | |
| PCDHF-73 | | 12 μg | |
| PCDHF-74 | | | 12 μg |
| PEI(Polysciences636951) | 60 μg | 60 μg | 60 μg |
| OPTI-MEMI(Gibco31985070) | 3 mL | 3 mL | 3 mL |

c. 48 hours after packaging, a cell supernatant is collected, and the lentivirus titer is detected after ultracentrifugation at 25,000 rpm. A detection method is as follows: 293T cells are infected with the collected lentivirus stock solution under a condition of the same gradient volume, and after 48 h, the percentage of GFP positive rate of the 293T cell is detected by a flow cytometry, according to a calculation formula: stock solution titer (TU/mL)=1.5×10E+05×293T cell GFP positive rate percentage/lentivirus stock solution volume μl×1000, the lentivirus stock solution titer is calculated.

2) CART Cell Preparation

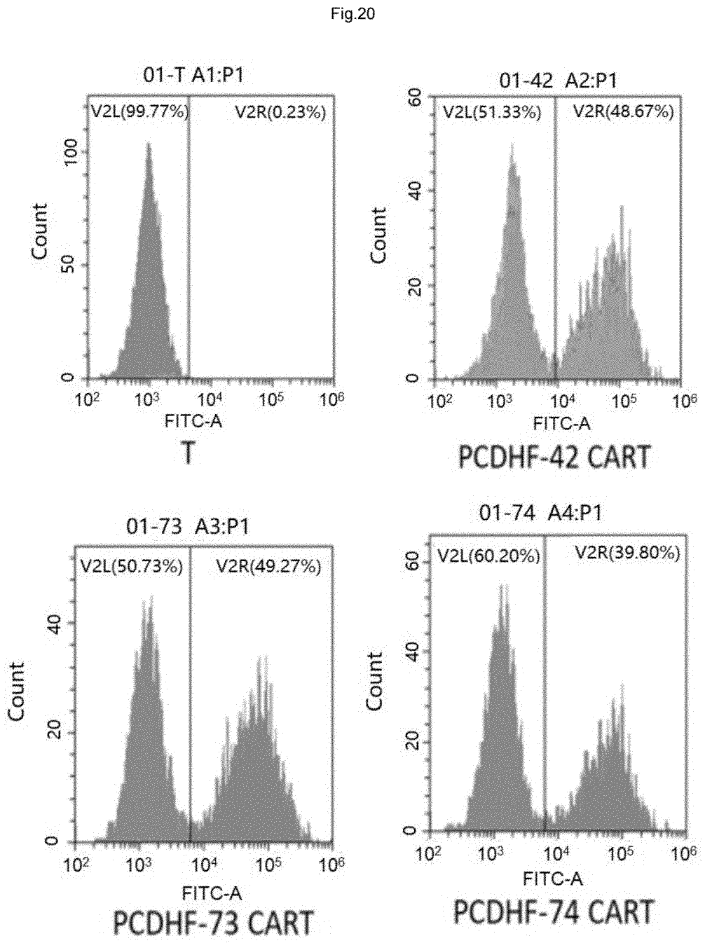

Ficoll lymphatic separation solution (Dakwin, AS1114546) is used to separate PBMC cells from blood (50 mL of volunteer blood), and T cells are separated and obtained by a magnetic bead positive selection method coupled with a CD3/CD28 antibody. T cells are infected at MOI=5:1 to prepare CART cells, and the CAR positive rate of the CART cells is determined by detecting the GFP positive rate of the CART cells after the CART cells are cultured for 7 days, as shown in FIG. 20.

3) In Vitro Functional Evaluation of CART Cell

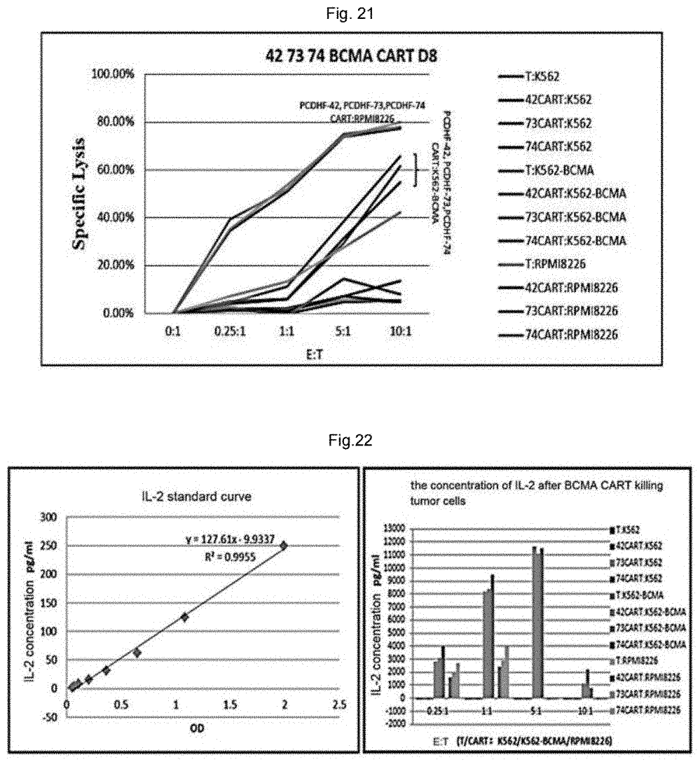

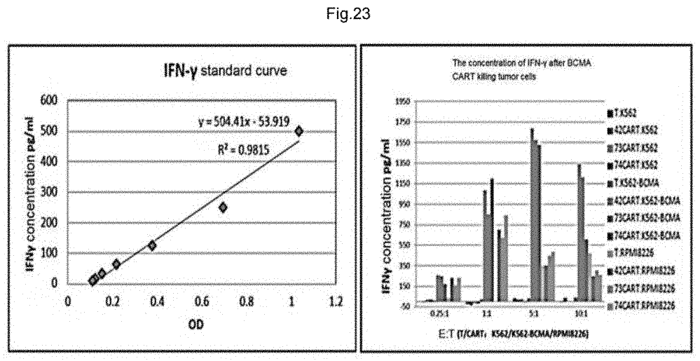

4 target cells K562, K562-BCMA, and RPMI8226 are respectively taken, each is 2×10E+06 cells, and firstly CytoCalcein™ Violet 550 is used to stain the target cells at 1×10E+05 cells/100 μl/well. Effector cells (CAR+CART, T cells are used as the control) and the above target cells are added to a 96-well plate at the ratio of 0.25:1, 1:1, 5:1 and 10:1 respectively and mixed uniformly, and the final volume is 200 μl. After 6 h of culture, the cells are mixed uniformly and centrifuged. A supernatant is detected by a Human IL-2 ELISA detection kit (invitrogen, REF 88-7025-88) and a Human IFN gamma ELISA kit (invitrogen, REF 88-7316-88) is used to detect the IL-2 and IFN-γ concentrations in each well, and a precipitation portion is resuspended with 100 μl of Annexin V Binding Buffer (Biolegend, B274722), it is centrifuged at 300 g for 5 min, after that, 3 μl of APC-Annexin V (Biolegend, Cat 640920) and 1.5 μl of PI dye (Biolegend, Cat 421301) are added, it is incubated in the dark for 15 min. 100 μl of Annexin V Binding Buffer is added to resuspend, after that, the Beckman Coulter flow cytometry is used to detect the apoptosis ratio of each target cell (results are shown in FIG. 21), and ELISA is used to detect the supernatant IL-2 and IFN-γ concentrations in each well (results are shown in FIG. 22 and FIG. 23). Herein, K562 is a BCMA-negative cell, and K562-BCMA and RPMI8226 are all BCMA-positive cells. The results show that PCDHF-73CART, PCDHF-74CART and PCDHF-42CART have the stronger specific killing on the BCMA-positive target cells, and the 3 CART cells have basically the same ability to kill the positive target cells, but have almost no killing effect on the BCMA-negative cells; and while PCDHF-42CART, PCDHF-73CART and PCDHF-74CART kill the BCMA-positive target cells, the secretion amounts of IL-2 and IFN-γ are at the same level. The comprehensive specific killing and factor secretion detection results show that humanized BCMA CART PCDHF-73CART and PCDHF-74CART and murine BCMA CART PCDHF-42CART have the same killing effect on the BCMA-positive target cells.

The sequences involved in the present disclosure are shown in the following table.

| SEQ | | |
|---|---|---|
| ID | | |
| NO: | Sequence name | Sequence |
| 1 | 5E2 CDR-VH1 | GYTFTSYVVH |
| 2 | 5E2 CDR-VH2 | IIPYNDDTK |
| 3 | 5E2 CDR-VH3 | ARW |
| 4 | 5E2 CDR-VL1 | SQSLLHSNGNTY |
| 5 | 5E2 CDR-VL2 | KVSNRFS |
| 6 | 5E2 CDR-VL3 | QITHIPFTF |
| 7 | 5E2 FR-H1 | EVQLQQSGPELIKPGASVKMSCKAS |
| 8 | 5E2 FR-H2 | WVKQKPGQGLEWIGY |
| 9 | 5E2 FR-H3 | YNEKFKGKATLTSDKSSSTAYMELS |
| | | SL |
| | | TSEDSAVYYC |
| 10 | 5E2 FR-H4 | DYDDGYFDYWGQGTTLTVSS |
| 11 | 5E2 FR-L1 | DVVMTQTPLSLPVTLGDQASISCRS |
| 12 | 5E2 FR-L2 | LHWYLQKPGQSPKLLIY |
| 13 | 5E2 FR-L3 | GVPDRFSGSGSGTDFTLKISRVEAE |
| | | DLGVYFCS |
| 14 | 5E2 FR-L4 | GSGTKLEIKR |
| 15 | 5E2 VH | EVQLQQSGPELIKPGASVKMSCKAS |
| | | GYTFTSYVVHWVKQKPGQGLEWIGY |
| | | IIPYNDDTKYNEKFKGKATLTSDKS |
| | | SSTAYMELSSLTSEDSAVYYCARW |
| | | DYDDGYFDYWGQGTTLTVSSDVVMT |
| | | QTPLSLPVTLGDQASISCRSSQSLL |
| 16 | 5E2 VL | HSNGNTYLHWYLQKPGQSPKLLIYK |
| | | VSNRFSGVPDRFSGSGSGTDFTLKI |
| | | SRVEAEDLGVYFCSQITHIPFTFGS |
| | | GTKLEIKR |
| 17 | scFv | CAGATCCAGCTGGTGCAGTCTGGCC |
| | sequence | CAGAGCTGAAGAAGCCCGGCGAGAC |
| | of C11D5.3 | CGTGAAGATCAGCTGCAAGGCCTCC |
| | | GGCTACACCTTCACAGACTATAGCA |
| | | TCAACTGGGTGAAGAGGGCCCCTGG |
| | | CAAGGGCCTGAAGTGGATGGGCTGG |
| | | ATCAATACCGAGACACGCGAGCCAG |
| | | CCTACGCCTATGACTTCCGGGGCAG |
| | | ATTCGCCTTTTCCCTGGAGACCTCT |
| | | GCCAGCACAGCCTACCTGCAGATCA |
| | | ACAATCTGAAGTACGAGGATACCGC |
| | | CACATATTTTTGCGCCCTGGACTAC |
| | | AGCTATGCCATGGATTATTGGGGCC |
| | | AGGGCACCTCCGTGACAGTGAGCTC |
| | | CGGAGGAGGAGGCTCCGGCGGCGGA |
| | | GGCTCTGGCGGCGGCGGCAGCGACA |
| | | TCGTGCTGACCCAGTCCCCAGCCTC |
| | | TCTGGCCATGTCCCTGGGCAAGCGG |
| | | GCCACAATCTCTTGTAGAGCCTCCG |
| | | AGTCTGTGAGCGTGATCGGCGCCCA |
| | | CCTGATCCACTGGTACCAGCAGAAG |
| | | CCTGGCCAGCCCCCTAAGCTGCTGA |
| | | TCTATCTGGCCAGCAACCTGGAGAC |
| | | CGGAGTGCCAGCACGGTTCTCCGGC |
| | | TCTGGCAGCGGCACAGACTTTACCC |
| | | TGACAATCGATCCTGTGGAGGAGGA |
| | | CGATGTGGCCATCTACTCTTGTCTG |
| | | CAGAGCAGGATCTTCCCACGCACCT |
| | | TTGGCGGCGGCACAAAGCTGGAGAT |
| | | CAAG |
| 18 | PCDHF | cgataccgtcgacctcgagacctag |
| | lentiviral | aaaaacatggagcaatcacaagtag |
| | vector | caatacagcagctaccaatgctgat |
| | | tgtgcctggctagaagcacaagagg |
| | | aggaggaggtgggttttccagtcac |
| | | acctcaggtacctttaagaccaatg |
| | | acttacaaggcagctgtagatctta |
| | | gccactttttaaaagaaaagggggg |
| | | actggaagggctaattcactcccaa |
| | | cgaagacaagatatccttgatctgt |
| | | ggatctaccacacacaaggctactt |
| | | ccctgattggcagaactacacacca |
| | | gggccagggatcagatatccactga |
| | | cctttggatggtgctacaagctagt |
| | | accagttgagcaagagaaggtagaa |
| | | gaagccaatgaaggagagaacaccc |
| | | gcttgttacaccctgtgagcctgca |
| | | tgggatggatgacccggagagagaa |
| | | gtattagagtggaggtttgacagcc |
| | | gcctagcatttcatcacatggcccg |
| | | agagctgcatccggactcgagataa |
| | | cttcgtataatgtatgctatacgaa |
| | | gttattccggactgtactgggtctc |
| | | tctggttagaccagatctgagcctg |
| | | ggagctctctggctaactagggaac |
| | | ccactgcttaagcctcaataaagct |
| | | tgccttgagtgcttcaagtagtgtg |
| | | tgcccgtctgttgtgtgactctggt |
| | | aactagagatccctcagaccctttt |
| | | agtcagtgtggaaaatctctagcag |
| | | ggcccgtttaaacccgctgatcagc |
| | | ctcgactgtgccttctagttgccag |
| | | ccatctgttgtttgcccctcccccg |
| | | tgccttccttgaccctggaaggtgc |
| | | cactcccactgtcctttcctaataa |
| | | aatgaggaaattgcatcgcattgtc |
| | | tgagtaggtgtcattctattctggg |
| | | gggtggggtggggcaggacagcaag |
| | | ggggaggattgggaagacaatagca |
| | | ggcatgtgagcaaaaggccagcaaa |
| | | aggccaggaaccgtaaaaaggccgc |
| | | gttgctggcgtttttccataggctc |
| | | cgcccccctgacgagcatcacaaaa |
| | | atcgacgctcaagtcagaggtggcg |
| | | aaacccgacaggactataaagatac |
| | | caggcgtttccccctggaagctccc |
| | | tcgtgcgctctcctgttccgaccct |
| | | gccgcttaccggatacctgtccgcc |
| | | tttctcccttcgggaagcgtggcgc |
| | | tttctcatagctcacgctgtaggta |
| | | tctcagttcggtgtaggtcgttcgc |
| | | tccaagctgggctgtgtgcacgaac |
| | | cccccgttcagcccgaccgctgcgc |
| | | cttatccggtaactatcgtcttgag |
| | | tccaacccggtaagacacgacttat |
| | | cgccactggcagcagccactggtaa |
| | | caggattagcagagcgaggtatgta |
| | | ggcggtgctacagagttcttgaagt |
| | | ggtggcctaactacggctacactag |
| | | aagaacagtatttggtatctgcgct |
| | | ctgctgaagccagttaccttcggaa |
| | | aaagagttggtagctcttgatccgg |
| | | caaacaaaccaccgctggtagcggt |
| | | ggtttttttgtttgcaagcagcaga |
| | | ttacgcgcagaaaaaaaggatctca |
| | | agaagatcctttgatcttttctacg |
| | | gggtctgacgctcagtggaacgaaa |
| | | actcacgttaagggattttggtcat |
| | | gagattatcaaaaaggatcttcacc |
| | | tagatccttttaaattaaaaatgaa |
| | | gttttaaatcaatctaaagtatata |
| | | tgagtaaacttggtctgacagttac |
| | | caatgcttaatcagtgaggcaccta |
| | | tctcagcgatctgtctatttcgttc |
| | | atccatagttgcctgactccccgtc |
| | | gtgtagataactacgatacgggagg |
| | | gcttaccatctggccccagtgctgc |
| | | aatgataccgcgagacccacgctca |
| | | ccggctccagatttatcagcaataa |
| | | accagccagccggaagggccgagcg |
| | | cagaagtggtcctgcaactttatcc |
| | | gcctccatccagtctattaattgtt |
| | | gccgggaagctagagtaagtagttc |
| | | gccagttaatagtttgcgcaacgtt |
| | | gttgccattgctacaggcatcgtgg |
| | | tgtcacgctcgtcgtttggtatggc |
| | | ttcattcagctccggttcccaacga |
| | | tcaaggcgagttacatgatccccca |
| | | tgttgtgcaaaaaagcggttagctc |
| | | cttcggtcctccgatcgttgtcaga |
| | | agtaagttggccgcagtgttatcac |
| | | tcatggttatggcagcactgcataa |
| | | ttctcttactgtcatgccatccgta |
| | | agatgcttttctgtgactggtgagt |
| | | actcaaccaagtcattctgagaata |
| | | gtgtatgcggcgaccgagttgctct |
| | | tgcccggcgtcaatacgggataata |
| | | ccgcgccacatagcagaactttaaa |
| | | agtgctcatcattggaaaacgttct |
| | | tcggggcgaaaactctcaaggatct |
| | | taccgctgttgagatccagttcgat |
| | | gtaacccactcgtgcacccaactga |
| | | tcttcagcatcttttactttcacca |
| | | gcgtttctgggtgagcaaaaacagg |
| | | aaggcaaaatgccgcaaaaaaggga |
| | | ataagggcgacacggaaatgttgaa |
| | | tactcatactcttcctttttcaata |
| | | ttattgaagcatttatcagggttat |
| | | tgtctcatgagcggatacatatttg |
| | | aatgtatttagaaaaataaacaaat |
| | | aggggttccgcgcacatttccccga |
| | | aaagtgccacctgacgtcgacggat |
| | | cgggagatctcccgatcccctatgg |
| | | tgcactctcagtacaatctgctctg |
| | | atgccgcatagttaagccagtatct |
| | | gctccctgcttgtgtgttggaggtc |
| | | gctgagtagtgcgcgagcaaaattt |
| | | aagctacaacaaggcaaggcttgac |
| | | cgacaattgcatgaagaatctgctt |
| | | agggttaggcgttttgcgctgcttc |
| | | gcgatgtacgggccagatatacgcg |
| | | ttgacattgattattgactagttat |
| | | taatagtaatcaattacggggtcat |
| | | tagttcatagcccatatatggagtt |
| | | ccgcgttacataacttacggtaaat |
| | | ggcccgcctggctgaccgcccaacg |
| | | acccccgcccattgacgtcaataat |
| | | gacgtatgttcccatagtaacgcca |
| | | atagggactttccattgacgtcaat |
| | | gggtggagtatttacggtaaactgc |
| | | ccacttggcagtacatcaagtgtat |
| | | catatgccaagtacgccccctattg |
| | | acgtcaatgacggtaaatggcccgc |
| | | ctggcattatgcccagtacatgacc |
| | | ttatgggactttcctacttggcagt |
| | | acatctacgtattagtcatcgctat |
| | | taccatggtgatgcggttttggcag |
| | | tacatcaatgggcgtggatagcggt |
| | | ttgactcacggggatttccaagtct |
| | | ccaccccattgacgtcaatgggagt |
| | | ttgttttggcaccaaaatcaacggg |
| | | actttccaaaatgtcgtaacaactc |
| | | cgccccattgacgcaaatgggcggt |
| | | aggcgtgtacggtgggaggtctata |
| | | taagcagcgcgttttgcctgtactg |
| | | ggtctctctggttagaccagatctg |
| | | agcctgggagctctctggctaacta |
| | | gggaacccactgcttaagcctcaat |
| | | aaagcttgccttgagtgcttcaagt |
| | | agtgtgtgcccgtctgttgtgtgac |
| | | tctggtaactagagatccctcagac |
| | | ccttttagtcagtgtggaaaatctc |
| | | tagcagtggcgcccgaacagggact |
| | | tgaaagcgaaagggaaaccagagga |
| | | gctctctcgacgcaggactcggctt |
| | | gctgaagcgcgcacggcaagaggcg |
| | | aggggcggcgactggtgagtacgcc |
| | | aaaaattttgactagcggaggctag |
| | | aaggagagagatgggtgcgagagcg |
| | | tcagtattaagcgggggagaattag |
| | | atcgcgatgggaaaaaattcggtta |
| | | aggccagggggaaagaaaaaatata |
| | | aattaaaacatatagtatgggcaag |
| | | cagggagctagaacgattcgcagtt |
| | | aatcctggcctgttagaaacatcag |
| | | aaggctgtagacaaatactgggaca |
| | | gctacaaccatcccttcagacagga |
| | | tcagaagaacttagatcattatata |
| | | atacagtagcaaccctctattgtgt |
| | | gcatcaaaggatagagataaaagac |
| | | accaaggaagctttagacaagatag |
| | | aggaagagcaaaacaaaagtaagac |
| | | caccgcacagcaagcggccgctgat |
| | | cttcagacctggaggaggagatatg |
| | | agggacaattggagaagtgaattat |
| | | ataaatataaagtagtaaaaattga |
| | | accattaggagtagcacccaccaag |
| | | gcaaagagaagagtggtgcagagag |
| | | aaaaaagagcagtgggaataggagc |
| | | tttgttccttgggttcttgggagca |
| | | gcaggaagcactatgggcgcagcgt |
| | | caatgacgctgacggtacaggccag |
| | | acaattattgtctggtatagtgcag |
| | | cagcagaacaatttgctgagggcta |
| | | ttgaggcgcaacagcatctgttgca |
| | | actcacagtctggggcatcaagcag |
| | | ctccaggcaagaatcctggctgtgg |
| | | aaagatacctaaaggatcaacagct |
| | | cctggggatttggggttgctctgga |
| | | aaactcatttgcaccactgctgtgc |
| | | cttggaatgctagttggagtaataa |
| | | atctctggaacagatttggaatcac |
| | | acgacctggatggagtgggacagag |
| | | aaattaacaattacacaagcttaat |
| | | acactccttaattgaagaatcgcaa |
| | | aaccagcaagaaaagaatgaacaag |
| | | aattattggaattagataaatgggc |
| | | aagtttgtggaattggtttaacata |
| | | acaaattggctgtggtatataaaat |
| | | tattcataatgatagtaggaggctt |
| | | ggtaggtttaagaatagtttttgct |
| | | gtactttctatagtgaatagagtta |
| | | ggcagggatattcaccattatcgtt |
| | | tcagacccacctcccaaccccgagg |
| | | ggacccgacaggcccgaaggaatag |
| | | aagaagaaggtggagagagagacag |
| | | agacagatccattcgattagtgaac |
| | | ggatcggcactgcgtgcgccaattc |
| | | tgcagacaaatggcagtattcatcc |
| | | acaattttaaaagaaaaggggggat |
| | | tggggggtacagtgcaggggaaaga |
| | | atagtagacataatagcaacagaca |
| | | tacaaactaaagaattacaaaaaca |
| | | aattacaaaaattcaaaattttcgg |
| | | gtttattacagggacagcagagatc |
| | | cagtttggttaattaacgtgaggct |
| | | ccggtgcccgtcagtgggcagagcg |
| | | cacatcgcccacagtccccgagaag |
| | | ttggggggaggggtcggcaattgaC |
| | | ccggtgcctagagaaggtggcgcgg |
| | | ggtaaactgggaaagtgatgtcgtg |
| | | tactggctccgcctttttcccgagg |
| | | gtgggggagaaccgtatataagtgc |
| | | agtagtcgccgtgaacgttcttttt |
| | | cgcaacgggtttgccgccagaacac |
| | | aggtaagtgccgtgtgtggttcccg |
| | | cgggcctggcctctttacgggttat |
| | | ggcccttgcgtgccttgaattactt |
| | | ccacctggctgcagtacgtgattct |
| | | tgatcccgagcttcgggttggaagt |
| | | gggtgggagagttcgaggccttgcg |
| | | cttaaggagccccttcgcctcgtgc |
| | | ttgagttgaggcctggcctgggcgc |
| | | tggggccgccgcgtgcgaatctggt |
| | | ggcaccttcgcgcctgtctcgctgc |
| | | tttcgataagtctctagccatttaa |
| | | aatttttgatgacctgctgcgacgc |
| | | tttttttctggcaagatagtcttgt |
| | | aaatgcgggccaagatctgcacact |
| | | ggtatttcggtttttggggccgcgg |
| | | gcggcgacggggcccgtgcgtccca |
| | | gcgcacatgttcggcgaggcggggc |
| | | ctgcgagcgcggccaccgagaatcg |
| | | gacgggggtagtctcaagctcgccg |
| | | gcctgctctggtgcctggcctcgcg |
| | | ccgccgtgtatcgccccgccctggg |
| | | cggcaaggctggcccggtcggcacc |
| | | agttgcgtgagcggaaagatggccg |
| | | cttcccggccctgctgcagggagct |
| | | caaaatggaggacgcggcgctcggg |
| | | agagcgggcgggtgagtcacccaca |
| | | caaaggaaaagggcctttccgtcct |
| | | cagccgtcgcttcatgtgactccac |
| | | tgagtaccgggcgccgtccaggcac |
| | | ctcgattagttctcgagcttttgga |
| | | gtacgtcgtctttaggttgggggga |
| | | ggggttttatgcgatggagtttccc |
| | | cacactgagtgggtggagactgaag |
| | | ttaggccagcttggcacttgatgta |
| | | attctccttggaatttgcccttttt |
| | | gagtttggatcttggttcattctca |
| | | agcctcagacagtggttcaaagttt |
| | | ttttcttccatttcaggtgtcgtga |
| | | gggatccccggaattcatcgatgcc |
| | | actaacttctccctgttgaaacaag |
| | | caggggatgtcgaagagaatcccgg |
| | | gccaatggtgagcaagggcgaggag |
| | | ctgttcaccggggtggtgcccatcc |
| | | tggtcgagctggacggcgacgtaaa |
| | | cggccacaagttcagcgtgtccggc |
| | | gagggcgagggcgatgccacctacg |
| | | gcaagctgaccctgaagttcatctg |
| | | caccaccggcaagctgcccgtgccc |
| | | tggcccaccctcgtgaccaccctga |
| | | cctacggcgtgcagtgcttcagccg |
| | | ctaccccgaccacatgaagcagcac |
| | | gacttcttcaagtccgccatgcccg |
| | | aaggctacgtccaggagcgcaccat |
| | | cttcttcaaggacgacggcaactac |
| | | aagacccgcgccgaggtgaagttcg |
| | | agggcgacaccctggtgaaccgcat |
| | | cgagctgaagggcatcgacttcaag |
| | | gaggacggcaacatcctggggcaca |
| | | agctggagtacaactacaacagcca |
| | | caacgtctatatcatggccgacaag |
| | | cagaagaacggcatcaaggtgaact |
| | | tcaagatccgccacaacatcgagga |
| | | cggcagcgtgcagctcgccgaccac |
| | | taccagcagaacacccccatcggcg |
| | | acggccccgtgctgctgcccgacaa |
| | | ccactacctgagcacccagtccgcc |
| | | ctgagcaaagaccccaacgagaagc |
| | | gcgatcacatggtcctgctggagtt |
| | | cgtgaccgccgccgggatcactctc |
| | | ggcatggacgagctgtacaagtaac |
| | | ttaaggccggccgacgcccttgacg |
| | | attttgacttagacatgctcccagc |
| | | cgatgcccttgacgactttgacctt |
| | | gatatgctgcctgctgacgctcttg |
| | | acgattttgaccttgacatgctccc |
| | | cgggtaactaagtaaggatcaattc |
| | | gatatcaagcttatcgataatcaac |
| | | ctctggattacaaaatttgtgaaag |
| | | attgactggtattcttaactatgtt |
| | | gctccttttacgctatgtggatacg |
| | | ctgctttaatgcctttgtatcatgc |
| | | tattgcttcccgtatggctttcatt |
| | | ttctcctccttgtataaatcctggt |
| | | tgctgtctctttatgaggagttgtg |
| | | gcccgttgtcaggcaacgtggcgtg |
| | | gtgtgcactgtgtttgctgacgcaa |
| | | cccccactggttggggcattgccac |
| | | cacctgtcagctcctttccgggact |
| | | ttcgctttccccctccctattgcca |
| | | cggcggaactcatcgccgcctgcct |
| | | tgcccgctgctggacaggggctcgg |
| | | ctgttgggcactgacaattccgtgg |
| | | tgttgtcggggaaatcatcgtcctt |
| | | tccttggctgctcgcctgtgttgcc |
| | | acctggattctgcgcgggacgtcct |
| | | tctgctacgtcccttcggccctcaa |
| | | tccagcggaccttccttcccgcggc |
| | | ctgctgccggctctgcggcctcttc |
| | | cgcgtcttcgccttcgccctcagac |
| | | gagtcggatctccctttgggccgcc |
| | | tccccgcat |
| 19 | 5F4CDR-VH1 | AGKWERWAH |
| 20 | 5F4CDR-VH2 | KPAYWTSA |
| 21 | 5F4CDR-VH3 | ATL |
| 22 | 5F4CDR-VL1 | RASQSVSSSYLA |
| 23 | 5F4CDR-VL2 | SDATGIP |
| 24 | 5F4CDR-VL3 | QQYGYPPSY |
| 25 | 5F4 FR-H1 | EVQLQQSGPELIKPGASVKMSCKAS |
| 26 | 5F4FR-H2 | WVKQKPGQGLEWIGY |
| 27 | 5F4FR-H3 | YNEKFKGKATLTSDKSSSTAYMELS |
| | | SLTSEDSAVYYC |
| 28 | 5F4FR-H4 | DYDDGYFDYWGQGTTLTVSS |
| 29 | 5F4FR-L1 | EIVLTQSPGTLSLSPGERATLSC |
| 30 | 5F4FR-L2 | WYQQKPGQAPRLLIYQAS |
| 31 | 5F4FR-L3 | DRFSGSGSGTDFTLTISRLEPEDFA |
| | | VYYC |
| 32 | 5F4FR-L4 | TFGQGTKVEIK |
| 33 | 5F4VH | EVQLQQSGPELIKPGASVKMSCKAS |
| | | AGKWERWAHWVKQKPGQGLEWIGYK |
| | | PAYWTSAYNEKFKGKATLTSDKSSS |
| | | TAYMELSSLTSEDSAVYYCATLDYD |
| | | DGYFDYWGQGTTLTVSS |
| 34 | 5F4VL | EIVLTQSPGTLSLSPGERATLSCRA |
| | | SQSVSSSYLAWYQQKPGQAPRLLIY |
| | | QASSDATGIPDRFSGSGSGTDFTLT |
| | | ISRLEPEDFAVYYCQQYGYPPSYTF |
| | | GQGTKVEIK |
| 35 | Linker | GGAGGAGGAGGCTCCGGCGGCGGAG |
| | peptide | GCTCTGGCGGCGGCGGCAGC |
| 36 | huVH1 | QVQLVQSGAEVKKPGASVKMSCKAS |
| | | GYTFTSYVVHWVRQAPGQGLEWIGYI |
| | | IPYNDDTKYNEKFKGKATLTSDKSS |
| | | STAYMELSSLRSEDTAVYYCARWDY |
| | | DDGYFDYWGQGTTVTVSS |
| 37 | huVH2 | QVQLVQSGAEVKKPGASVKMSCKAS |
| | | GYTFTSYVVHWVRQAPGQGLEWIGYI |
| | | IPYNDDTKYNEKFKGRVTLTSDKST |
| | | STAYMELSSLRSEDTAVYYCARWDY |
| | | DDGYFDYWGQGTTVTVSS |
| 38 | huVL1 | DVVMTQSPLSLPVTLGQPASISCRSS |
| | | QSLLHSNGNTYLHWYLQKPGQSPQL |
| | | LIYKVSNRFSGVPDRFSGSGSGTDF |
| | | TLKISRVEAEDVGVYFCSQITHIPF |
| | | TFGQGTKLEIK |
| 39 | huVL2 | DVVMTQTPLSLSVTPGQPASISCKSS |
| | | QSLLHSNGNTYLHWYLQKPGQSPQL |
| | | LIYKVSNRFSGVPDRFSGSGSGTDF |
| | | TLKISRVEAEDVGVYFCSQITHIPF |
| | | TFGQGTKLEIK |
| 40 | huVL3 | DIVMTQTPLSLSVTPGQPASISCKS |
| | | SQSLLHSNGNTYLHWYLQKPGQPPQ |
| | | LLIYKVSNRFSGVPDRFSGSGSGTD |
| | | FTLKISRVEAEDVGVYYCSQITHIP |
| | | FTFGQGTKLEIKR |
| 41 | huVH1-FR-H1 | QVQLVQSGAEVKKPGASVKMSCKAS |
| 42 | huVH1-FR-H2 | WVRQAPGQGLEWIGY |
| 43 | huVH1-FR-H3 | YNEKFKGKATLTSDKSSSTAYMELS |
| | | SLRSEDTAVYYC |
| 44 | huVH1-FR-H4 | DYDDGYFDYWGQGTTVTVSS |
| 45 | huVH2-FR-H1 | QVQLVQSGAEVKKPGASVKMSCKAS |
| 46 | huVH2-FR-H2 | WVRQAPGQGLEWIGY |
| 47 | huVH2-FR-H3 | YNEKFKGRVTLTSDKSTSTAYMELS |
| | | SLRSEDTAVYYC |
| 48 | huVH2-FR-H4 | DYDDGYFDYWGQGTTVTVSS |
| 49 | huVL1-FR-L1 | DVVMTQSPLSLPVTLGQPASISCRS |
| 50 | huVL1-FR-L2 | LHWYLQKPGQSPQLLIY |
| 51 | huVL1-FR-L3 | VPDRFSGSGSGTDFTLKISRVEAED |
| | | VGVYFCS |
| 52 | huVL1-FR-L4 | GQGTKLEIK |
| 53 | HuVL2-FR-L1 | DVVMTQTPLSLSVTPGQPASISCKS |
| 54 | HuVL2-FR-L2 | LHWYLQKPGQSPQLLIY |
| 55 | HuVL2-FR-L3 | VPDRFSGSGSGTDFTLKISRVEAED |
| | | VGVYFCS |
| 56 | HuVL2-FR-L4 | GQGTKLEIK |
| 57 | HuVL3-FR-L1 | DIVMTQTPLSLSVTPGQPASISCKS |
| 58 | HuVL3-FR-L2 | LHWYLQKPGQPPQLLIY |
| 59 | HuVL3-FR-L3 | VPDRFSGSGSGTDFTLKISRVEAED |
| | | VGVYYCS |
| 60 | HuVL3-FR-L4 | GQGTKLEIKR |

Technical features of the above embodiments may be combined arbitrarily. For brevity of the description, all possible combinations of the technical features in the above embodiments are not described. However, as long as there is no contradiction between the combinations of these technical features, all should be regarded as a scope described in the description.

The above embodiments only represent several implementation modes of the present disclosure, and the descriptions thereof are more specific and detailed, but should not be construed as limitation to a scope of the disclosure patent. It should be pointed out that for those skilled in the art, without departing from the concept of the present disclosure, a plurality of modifications and improvements may also be made, and these all belong to a scope of protection of the present disclosure. Therefore, the scope of protection of the disclosure patent should be subject to the appended claims.

What is claimed is:

1. A B-cell maturation antigen (BCMA) antibody or an antigen-binding fragment thereof, wherein it is capable of specifically binding to BCMA, and selected from a) and/or b):
a) comprising heavy chain complementarity determining regions CDR-VH1, CDR-VH2, and CDR-VH3 of which amino acid sequences are as shown in SEQ ID NO: 1˜3, and light chain complementarity determining regions CDR-VL1, CDR-VL2, and CDR-VL3 of which amino acid sequences as shown in SEQ ID NO: 4˜6; and
b) comprising heavy chain complementarity determining regions CDR-VH1, CDR-VH2, and CDR-VH3 of which amino acid sequences are as shown in SEQ ID NO: 19˜21, and light chain complementarity determining regions CDR-VL1, CDR-VL2, and CDR-VL3 of which amino acid sequences as shown in SEQ ID NO: 22˜24.

2. The antibody or antigen-binding fragment thereof according to claim 1, wherein a further comprises heavy chain framework regions FR-H1, FR-H2, FR-H3 and FR-H4 of which sequences are as shown in SEQ ID NO: 7˜10, SEQ ID NO: 41˜44, or SEQ ID NO: 45˜48; and/or light chain framework regions FR-L1, FR-L2, FR-L3 and FR-L4 of which sequences are as shown in SEQ ID NO: 11˜14, SEQ ID NO: 49˜52, SEQ ID NO: 53˜56 or SEQ ID NO: 57˜60; and
b further comprises heavy chain framework regions FR-H1, FR-H2, FR-H3 and FR-H4 of which sequences are as shown in SEQ ID NO: 25˜28; and/or light chain framework regions FR-L1, FR-L2, FR-L3 and FR-L4 of which sequences are as shown in SEQ ID NOs: 29˜32.

3. The antibody or antigen-binding fragment thereof according to claim 1, wherein the antibody is one of F(ab′)₂, Fab, Fv, scFv and a bispecific antibody.

4. The antibody or antigen-binding fragment thereof according to claim 1, wherein the antibody has a constant region, and a constant region sequence is selected from a sequence of any one of constant regions of IgG1, IgG2, IgG3, IgG4, IgA, IgM, IgE, and IgD.

5. A chimeric antigen receptor, wherein it contains the antibody according to claim 1.

6. An isolated nucleic acid, wherein it encodes the antibody according to claim 1.

7. A vector, wherein it contains the isolated nucleic acid according to claim 6.

8. A host cell, wherein it contains the vector according to claim 7.

9. An immune cell, wherein it contains the chimeric antigen receptor according to claim 5.

10. A pharmaceutical composition, wherein it comprises the antibody according to claim 1, and one or more of a pharmaceutically acceptable excipient, diluent or carrier.

11. The antibody or antigen-binding fragment thereof according to claim 4, wherein the species source of the constant region is independently selected from bovine, equine, dairy cow, pig, sheep, goat, rat, mouse, dog, cat, rabbit, camel, donkey, deer, mink, chicken, duck, goose, turkey, cockfight or human.

12. The chimeric antigen receptor according to claim 5, wherein the chimeric antigen receptor comprises one or more elements selected from a group consisting of the following components: a leader peptide, a linker sequence, a transmembrane domain, a costimulatory domain and a signal conduction domain.

13. An isolated nucleic acid, wherein it encodes the chimeric antigen receptor according to claim 6.

14. The vector according to claim 7, wherein the vector is a lentiviral vector.

15. The vector according to claim 14, wherein a nucleotide sequence of the lentiviral vector is shown in SEQ ID NO: 18.

16. The immune cell according to claim 9, wherein the immune cell is a T cell, a tumor infiltrating lymphocyte, a NK cell, a dendritic cell or a NK-T cell.

17. The immune cell according to claim 9, wherein the immune cell is an autologous T cell or an allogeneic T cell.

18. A pharmaceutical composition, wherein it comprises the immune cell according to claim 9, and one or more of a pharmaceutically acceptable excipient, diluent or carrier.

19. A method for treating multiple myeloma in a subject in need thereof, the method comprising:
a) providing the pharmaceutical composition; as well as
b) administering a therapeutically effective amount of the pharmaceutical composition to the subject.


SEQUENCE LISTING (60 sequences)

SEQ ID NO: 1 - protein, 10 aa, Artificial Sequence
  artificially synthesized
GYTFTSYVVH

SEQ ID NO: 2 - protein, 9 aa, Artificial Sequence
  artificially synthesized
IIPYNDDTK

SEQ ID NO: 3 - protein, 3 aa, Artificial Sequence
  artificially synthesized
ARW

SEQ ID NO: 4 - protein, 12 aa, Artificial Sequence
  artificially synthesized
SQSLLHSNGNTY

SEQ ID NO: 5 - protein, 7 aa, Artificial Sequence
  artificially synthesized
KVSNRFS

SEQ ID NO: 6 - protein, 9 aa, Artificial Sequence
  artificially synthesized
QITHIPFTF

SEQ ID NO: 7 - protein, 25 aa, Artificial Sequence
  artificially synthesized
EVQLQQSGPELIKPGASVKMSCKAS

SEQ ID NO: 8 - protein, 15 aa, Artificial Sequence
  artificially synthesized
WVKQKPGQGLEWIGY

SEQ ID NO: 9 - protein, 37 aa, Artificial Sequence
  artificially synthesized
YNEKFKGKATLTSDKSSSTAYMELSSLTSEDSAVYYC

SEQ ID NO: 10 - protein, 20 aa, Artificial Sequence
  artificially synthesized
DYDDGYFDYWGQGTTLTVSS

SEQ ID NO: 11 - protein, 25 aa, Artificial Sequence
  artificially synthesized
DVVMTQTPLSLPVTLGDQASISCRS

SEQ ID NO: 12 - protein, 17 aa, Artificial Sequence
  artificially synthesized
LHWYLQKPGQSPKLLIY

SEQ ID NO: 13 - protein, 33 aa, Artificial Sequence
  artificially synthesized
GVPDRFSGSGSGTDFTLKISRVEAEDLGVYFCS

SEQ ID NO: 14 - protein, 10 aa, Artificial Sequence
  artificially synthesized
GSGTKLEIKR

SEQ ID NO: 15 - protein, 119 aa, Artificial Sequence
  artificially synthesized
EVQLQQSGPELIKPGASVKMSCKASGYTFTSYVVHWVKQKPGQGLEWIGYIIPYNDDTKYNEKFKGKATLTSDKSSSTAYMELSSLTSEDSAVYYCARWDYDDGYFDYWGQGTTLTVSS

SEQ ID NO: 16 - protein, 113 aa, Artificial Sequence
  artificially synthesized
DVVMTQTPLSLPVTLGDQASISCRSSQSLLHSNGNTYLHWYLQKPGQSPKLLIYKVSNRFSGVPDRFSGSGSGTDFTLKISRVEAEDLGVYFCSQITHIPFTFGSGTKLEIKR

SEQ ID NO: 17 - DNA, 729 bp, Artificial Sequence
  artificially synthesized
cagatccagctggtgcagtctggcccagagctgaagaagcccggcgagaccgtgaagatcagctgcaaggcctccggctacaccttcacagactatagcatcaactgggtgaagagggcccctggcaagggcctgaagtggatgggctggatcaataccgagacacgcgagccagcctacgcctatgacttccggggcagattcgccttttccctggagacctctgccagcacagcctacctgcagatcaacaatctgaagtacgaggataccgccacatatttttgcgccctggactacagctatgccatggattattggggccagggcacctccgtgacagtgagctccggaggaggaggctccggcggcggaggctctggcggcggcggcagcgacatcgtgctgacccagtccccagcctctctggccatgtccctgggcaagcgggccacaatctcttgtagagcctccgagtctgtgagcgtgatcggcgcccacctgatccactggtaccagcagaagcctggccagccccctaagctgctgatctatctggccagcaacctggagaccggagtgccagcacggttctccggctctggcagcggcacagactttaccctgacaatcgatcctgtggaggaggacgatgtggccatctactcttgtctgcagagcaggatcttcccacgcacctttggcggcggcacaaagctggagatcaag

SEQ ID NO: 18 - DNA, 8134 bp, Artificial Sequence
  artificially synthesized
cgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactcgagataacttcgtataatgtatgctatacgaagttattccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacgtgaggctccggtgcccgtcagtgggcagagcgcacatcgcccacagtccccgagaagttggggggaggggtcggcaattgacccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctcgccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagggatccccggaattcatcgatgccactaacttctccctgttgaaacaagcaggggatgtcgaagagaatcccgggccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacttaaggccggccgacgcccttgacgattttgacttagacatgctcccagccgatgcccttgacgactttgaccttgatatgctgcctgctgacgctcttgacgattttgaccttgacatgctccccgggtaactaagtaaggatcaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcat

SEQ ID NO: 19 - protein, 9 aa, Artificial Sequence
  artificially synthesized
AGKWERWAH

SEQ ID NO: 20 - protein, 8 aa, Artificial Sequence
  artificially synthesized
KPAYWTSA

SEQ ID NO: 21 - protein, 3 aa, Artificial Sequence
  artificially synthesized
ATL

SEQ ID NO: 22 - protein, 12 aa, Artificial Sequence
  artificially synthesized
RASQSVSSSYLA

SEQ ID NO: 23 - protein, 7 aa, Artificial Sequence
  artificially synthesized
SDATGIP

SEQ ID NO: 24 - protein, 9 aa, Artificial Sequence
  artificially synthesized
QQYGYPPSY

SEQ ID NO: 25 - protein, 25 aa, Artificial Sequence
  artificially synthesized
EVQLQQSGPELIKPGASVKMSCKAS

SEQ ID NO: 26 - protein, 15 aa, Artificial Sequence
  artificially synthesized
WVKQKPGQGLEWIGY

SEQ ID NO: 27 - protein, 37 aa, Artificial Sequence; identical to SEQ ID NO: 9
  artificially synthesized

SEQ ID NO: 28 - protein, 20 aa, Artificial Sequence
  artificially synthesized
DYDDGYFDYWGQGTTLTVSS

SEQ ID NO: 29 - protein, 23 aa, Artificial Sequence
  artificially synthesized
EIVLTQSPGTLSLSPGERATLSC

SEQ ID NO: 30 - protein, 18 aa, Artificial Sequence
  artificially synthesized
WYQQKPGQAPRLLIYQAS

SEQ ID NO: 31 - protein, 29 aa, Artificial Sequence
  artificially synthesized
DRFSGSGSGTDFTLTISRLEPEDFAVYYC

SEQ ID NO: 32 - protein, 11 aa, Artificial Sequence
  artificially synthesized
TFGQGTKVEIK

SEQ ID NO: 33 - protein, 117 aa, Artificial Sequence
  artificially synthesized
EVQLQQSGPELIKPGASVKMSCKASAGKWERWAHWVKQKPGQGLEWIGYKPAYWTSAYNEKFKGKATLTSDKSSSTAYMELSSLTSEDSAVYYCATLDYDDGYFDYWGQGTTLTVSS

SEQ ID NO: 34 - protein, 109 aa, Artificial Sequence
  artificially synthesized
EIVLTQSPGTLSLSPGERATLSCRASQSVSSSYLAWYQQKPGQAPRLLIYQASSDATGIPDRFSGSGSGTDFTLTISRLEPEDFAVYYCQQYGYPPSYTFGQGTKVEIK

SEQ ID NO: 35 - DNA, 45 bp, Artificial Sequence
  artificially synthesized
ggaggaggaggctccggcggcggaggctctggcggcggcggcagc

SEQ ID NO: 36 - protein, 119 aa, Artificial Sequence
  artificially synthesized
QVQLVQSGAEVKKPGASVKMSCKASGYTFTSYVVHWVRQAPGQGLEWIGYIIPYNDDTKYNEKFKGKATLTSDKSSSTAYMELSSLRSEDTAVYYCARWDYDDGYFDYWGQGTTVTVSS

SEQ ID NO: 37 - protein, 119 aa, Artificial Sequence
  artificially synthesized
QVQLVQSGAEVKKPGASVKMSCKASGYTFTSYVVHWVRQAPGQGLEWIGYIIPYNDDTKYNEKFKGRVTLTSDKSTSTAYMELSSLRSEDTAVYYCARWDYDDGYFDYWGQGTTVTVSS

SEQ ID NO: 38 - protein, 112 aa, Artificial Sequence
  artificially synthesized
DVVMTQSPLSLPVTLGQPASISCRSSQSLLHSNGNTYLHWYLQKPGQSPQLLIYKVSNRFSGVPDRFSGSGSGTDFTLKISRVEAEDVGVYFCSQITHIPFTFGQGTKLEIK

SEQ ID NO: 39 - protein, 112 aa, Artificial Sequence
  artificially synthesized
DVVMTQTPLSLSVTPGQPASISCKSSQSLLHSNGNTYLHWYLQKPGQSPQLLIYKVSNRFSGVPDRFSGSGSGTDFTLKISRVEAEDVGVYFCSQITHIPFTFGQGTKLEIK

SEQ ID NO: 40 - protein, 113 aa, Artificial Sequence
  artificially synthesized
DIVMTQTPLSLSVTPGQPASISCKSSQSLLHSNGNTYLHWYLQKPGQPPQLLIYKVSNRFSGVPDRFSGSGSGTDFTLKISRVEAEDVGVYYCSQITHIPFTFGQGTKLEIKR

SEQ ID NO: 41 - protein, 25 aa, Artificial Sequence
  artificially synthesized
QVQLVQSGAEVKKPGASVKMSCKAS

SEQ ID NO: 42 - protein, 15 aa, Artificial Sequence
  artificially synthesized
WVRQAPGQGLEWIGY

SEQ ID NO: 43 - protein, 37 aa, Artificial Sequence
  artificially synthesized
YNEKFKGKATLTSDKSSSTAYMELSSLRSEDTAVYYC

SEQ ID NO: 44 - protein, 20 aa, Artificial Sequence
  artificially synthesized
DYDDGYFDYWGQGTTVTVSS

SEQ ID NO: 45 - protein, 25 aa, Artificial Sequence
  artificially synthesized
QVQLVQSGAEVKKPGASVKMSCKAS

SEQ ID NO: 46 - protein, 15 aa, Artificial Sequence
  artificially synthesized
WVRQAPGQGLEWIGY

SEQ ID NO: 47 - protein, 37 aa, Artificial Sequence
  artificially synthesized
YNEKFKGRVTLTSDKSTSTAYMELSSLRSEDTAVYYC

SEQ ID NO: 48 - protein, 20 aa, Artificial Sequence
  artificially synthesized
DYDDGYFDYWGQGTTVTVSS

SEQ ID NO: 49 - protein, 25 aa, Artificial Sequence
  artificially synthesized
DVVMTQSPLSLPVTLGQPASISCRS

SEQ ID NO: 50 - protein, 17 aa, Artificial Sequence
  artificially synthesized
LHWYLQKPGQSPQLLIY

SEQ ID NO: 51 - protein, 32 aa, Artificial Sequence
  artificially synthesized
VPDRFSGSGSGTDFTLKISRVEAEDVGVYFCS

SEQ ID NO: 52 - protein, 9 aa, Artificial Sequence
  artificially synthesized
GQGTKLEIK

SEQ ID NO: 53 - protein, 25 aa, Artificial Sequence
  artificially synthesized
DVVMTQTPLSLSVTPGQPASISCKS

SEQ ID NO: 54 - protein, 17 aa, Artificial Sequence
  artificially synthesized
LHWYLQKPGQSPQLLIY

SEQ ID NO: 55 - protein, 32 aa, Artificial Sequence; identical to SEQ ID NO: 51
  artificially synthesized

SEQ ID NO: 56 - protein, 9 aa, Artificial Sequence
  artificially synthesized
GQGTKLEIK

SEQ ID NO: 57 - protein, 25 aa, Artificial Sequence
  artificially synthesized
DIVMTQTPLSLSVTPGQPASISCKS

SEQ ID NO: 58 - protein, 17 aa, Artificial Sequence
  artificially synthesized
LHWYLQKPGQPPQLLIY

SEQ ID NO: 59 - protein, 32 aa, Artificial Sequence
  artificially synthesized
VPDRFSGSGSGTDFTLKISRVEAEDVGVYYCS

SEQ ID NO: 60 - protein, 10 aa, Artificial Sequence
  artificially synthesized
GQGTKLEIKR